(19) United States
(12) Patent Application Publication
(10) Pub. No.: US 2008/0132500 A1
(43) Pub. Date: Jun. 5, 2008

(54) Antibiotic compounds

(76) Inventors: Kun Liu, Edison, NJ (US); Peter T. Meinke, Scotch Plains, NJ (US); James F. Dropinski, Colts Neck, NJ (US); Libo Xu, Bridgewater, NJ (US)

Correspondence Address: MERCK AND CO., INC, P O BOX 2000, RAHWAY, NJ, 07065-0907, US

(21) Appl. No.: 11/975,537

(22) Filed: Oct. 19, 2007

Related U.S. Application Data
(60) Provisional application No. 60/853,393, filed on Oct. 20, 2006.

(51) Int. Cl. A61K 31/4365; C07D 419/14; A61K 31/496; A61K 31/5377; A61K 31/497; A61P 31/04

(52) U.S. Cl. 514/233.2; 540/455; 514/301; 514/256; 514/254.11

(57) ABSTRACT

Certain water-soluble thiazolyl peptides are antibiotic capable of treating serious bacterial infections in mammals, and particularly, in humans. Some of the analogs can also be employed as versatile intermediates for the preparation of new derivatives with useful antibacterial activity.

This application claims the benefit of U.S. Provisional Application No. 60/853,393, filed Oct. 20, 2006, the disclosure of which is hereby incorporated by reference in its entirety.

### BACKGROUND OF THE INVENTION

Infections caused by bacteria are a growing medical concern as many of these bacteria are resistant to various antibiotics. Such microbes include Staphylococcus aureus, Staphylococcus hemolyticus, Pediococcus spp., and Streptococcus pyogenes, Streptococcus pneumoniae, Pseudomonas aeruginosa, Vibrio cholerae, Vibrio parahemolyticus, Actinobacter calcoaeticus, Stenotrophomonas maltophilia.

Many thiazolyl peptide antibiotics exhibit potent antibacterial activity against a variety of Gram-positive bacteria, including multiple drug-resistant strains. Their poor water solubility severely limits their usage as therapeutic agents.

The present invention relates to novel water-soluble thiazolyl peptide antibiotics capable of treating serious bacterial infections in mammals, and particularly, in humans. Some of these analogs can also be versatile intermediates for the preparation of new derivatives with useful antibacterial activity. Many of the novel thiazolyl peptide antibiotics of the present invention show much improved aqueous solubility over previously disclosed antibiotics (see WO 2004/004646, WO 2002/14354, WO 2002/13834, WO 2000/68413, WO 200014100, WO 2000/03722, WO 2002/66046 and PCT US2005/33326, filed Sep. 16, 2005). While some methods have been reported to improve the aqueous solubility of thiazolyl peptide antibiotics [see P. Hmciar et al., J. Org. Chem. 2002, 67(25), 8789-8793; B. Naidu, et al., Bioorganic & Med. Chem. Ltrs. (2004), 14(22), 5573-5577; M. Pucci, et al., Antimicrobial Agts. And Chemo., (2004), 48(10), 3697-3701; B. Naidu, et al, Tetrahedron Letters (2004), 45(17), 3531, and Tetrahedron Letters (2004), 45(5), 1059-1063; M. D. Lee et al., J. Antibiotics August 1994, Vol. 47 No. 8 pages 901-908; T. Otani et al., J. Antibiotics 1998, Vol. 51 No. 8, pages 715-721; and M. D. Lee et al., J. Antibiotics 1994, Vol. 47 No. 8 pages 894-900], the current invention uses a different approach which utilizes the novel intermediates derived from natural products. The antibiotics of this invention thus comprise an important contribution to therapy for treating infections which are resistant to various known antibiotics.

The primary amines and aldehydes of the claimed invention can be derived from thiazolyl antibiotics such as thiostrepton, GE2270A, A10255, S 54832, promothiocin, thioactin, siomycins, berninamycin, thiopeptin, thiazomycin, nocathiacins, glycothiohexide, and nosiheptide.

### SUMMARY OF THE INVENTION

This invention is concerned with novel water soluble thiazolyl-peptide antibiotics of the formula I:

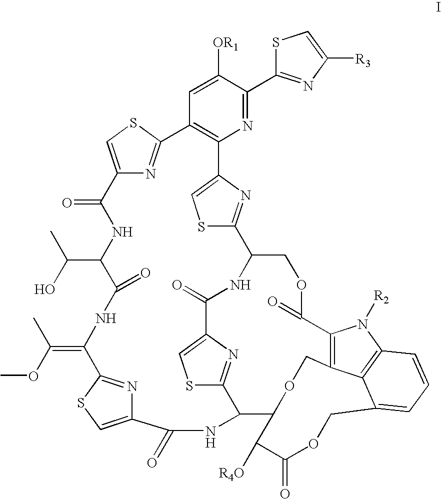

or a pharmaceutically acceptable salt, ester, enantiomer, diastereomer or mixture thereof, wherein:

R independently represents hydrogen, and C₁₋₁₂ alkyl;

R₁ represents hydrogen, C₁₋₆ alkyl, C₃₋₆ cycloalkyl, —(CH₂)ₙC₅₋₁₀ heterocyclyl, and —C(O)NR(CH₂)ₙC₅₋₁₀ heterocyclyl;

R₂ represents R₁ and OR₁;

R₃ represents —CH₂NR₅R_{6,} or C(O)H;

R₄ represents

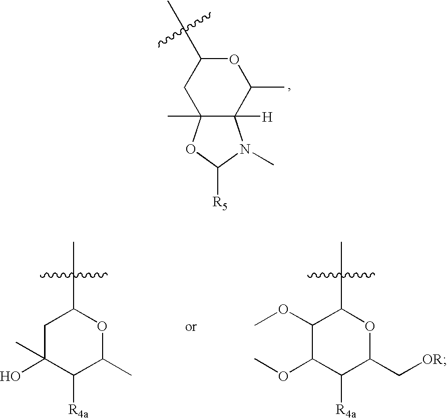

R₄a represents N(R)₂;

R₅ and R₆ independently represent hydrogen, C₁₋₁₂ alkyl, —C(═NH)N(R₁)_{2,}—(CH₂)ₙC₅₋₁₀ heterocyclyl, —(CH₂)ₙNR₇R_{8,}—(CH₂)ₙNR(CH₂)ₙNR₇R_{8,}—-(CH₂)ₙNR(CH₂)ₙC₅₋₁₀ heterocyclyl, —(CH₂)ₙC(R)₂C₅₋₁₀ heterocyclyl, —(CH₂)ₙC₅₋₁₀ aryl, —(CH₂)ₙ(O(CH₂)₂)₁₋₆R₉, —(CHR)ₙNHC(O)(CH₂)ₙNR₇R_{8,}—(CH₂)ₙS(O)ₚ(CH₂)ₙC₅₋₁₀ heterocyclyl, —(CH₂)ₙCHR₇CF_{3,} —C(O)C₁₋₆ alkyl, —C(O)CF_{3,}—C(O)(C(R)₂)ₙNR₁R_{7,}—C(O)NR(CH₂)ₙC₅₋₁₀ heterocyclyl, —C(O)NR(CH₂)ₙNR₇R_{8,}—C(O)C(R)₂NHC(O)(CH₂)ₙNR₇R_{8,}—C(O)CHR₇(CH₂)ₙC(O)NR₁R₁, —C(O)C(O)NR₁ R_{1,}—C(O)(CH₂)ₙC₅₋₁₀ heterocyclyl, —C(R)₂(CH₂)ₙNHC(O)N(CH₂)ₙC₅₋₁₀ heterocyclyl, —C(R)₂(CH₂)ₙOR, said aryl, and heterocyclyl optionally substituted with one or more groups of R^{a}; said alkyl optionally substituted with 1 to 6 hydroxy and/or optionally substituted by one or more groups of R^{a}; or

R₅ and R₆ together with the nitrogen atom they are attached form a 5 to 10 heterocyclic ring optionally containing 1 to 2 additional heteroatoms selected from the group consisting of N, S and O and optionally substituted with one or more groups of R^{a};

R₇ and R₈ independently represent hydrogen, hydroxyl, C₁₋₆ alkoxy, C₁₋₁₂ alkyl, —N(R)₂ —(CH₂)ₙNR₅R_{6,}—(CH₂)ₙC₅₋₁₀ heterocyclyl, —(CH₂)ₙC₆₋₁₀ aryl, —(CH₂)ₙOR, —C(O)R, —C(O)C₅₋₁₀ heterocyclyl, —C(O)NH(CH₂)ₙC₅₋₁₀ heterocyclyl, —C(O)(CH₂)ₙN(R)_{2,} said aryl, and heterocyclyl optionally substituted with one or more groups of R^{a}; said alkyl optionally substituted with 1 to 6 hydroxyl and/or optionally substituted by one to more groups of R^{a} or

R₇ and R₈ together with the nitrogen atom they are attached form a 5 to 10 membered heterocyclic ring optionally containing 1 to 2 additional heteroatoms selected from the group consisting of N, S and O and optionally substituted with one or more groups of R^{a}; or

R₇ and R₈ together with the carbon atom they are attached form a 3 to 10 membered carbocyclic ring optionally and optionally substituted with one or more groups of R^{a};

R₉ represents hydrogen, C₁₋₆ alkyl, (CH₂)ₙC₅₋₁₀ heterocyclyl, —C(O)OR, CN, OR, said alkyl and heterocyclyl optionally substituted with one or more groups of R^{a};

R^{a} represents hydrogen, halogen, (CH₂)ₙOR, CF_{3,} NHC(O)R, (CH₂)ₙC(O)OR, (CH₂)ₙC(O)NR₇R_{8,} (CH₂)ₙC₅₋₁₀ heterocyclyl, SO₂NR₅R_{6,} (CH₂)C₆₋₁₀ aryl, N(R)_{2,} NO_{2,} CN, —OP(O)(OR)_{2,} (C₁₋₆ alkyl)O—, (aryl)O—, (C₁₋₆ alkyl)S(O)₀₋₂—, C₁₋₁₂ alkyl, said alkyl, heterocyclyl, and aryl optionally substituted with 1 to 4 groups selected from the group consisting of C₁₋₆ alkyl, (CH₂)ₙOR, (CH₂)ₙN(R)_{2,}—O—; and

n represent 0-6, and p represents 0, 1 or 2.

### DETAILED DESCRIPTION OF THE INVENTION

The invention is described herein in detail using the terms defined below unless otherwise specified.

The compounds of the present invention may have asymmetric centers, chiral axes and chiral planes, and occur as racemates, racemic mixtures, and as individual diastereomers, with all possible isomers, including optical isomers, being included in the present invention. (See E. L. Eliel and S. H. Wilen Stereochemistry of Carbon Compounds (John Wiley and Sons, New York 1994), in particular pages 1119-1190).

When any variable (e.g. aryl, heterocycle, R_{4,} R₁, etc.) occurs more than one time in any constituent, its definition on each occurrence is independent at every other occurrence. Also, combinations of substituents/or variables are permissible only if such combinations result in stable compounds.

The term “alkyl” refers to a monovalent alkane (hydrocarbon) derived radical containing from 1 to 15 carbon atoms unless otherwise defined. It may be straight or branched. Preferred alkyl groups include lower alkyls which have from 1 to 6 carbon atoms such as methyl, ethyl, propyl, isopropyl, butyl and t-butyl. When substituted, alkyl groups may be substituted with up to 5 substituent groups, selected from the groups as herein defined, at any available point of attachment. When the alkyl group is said to be substituted with an alkyl group, this is used interchangeably with “branched alkyl group”.

Cycloalkyl is a species of alkyl containing from 3 to 15 carbon atoms, without alternating or resonating double bonds between carbon atoms. It may contain from 1 to 4 rings which are fused. Preferred cycloalkyl groups are cyclopropyl, cyclobutyl, cyclopentyl and cyclohexyl. When substituted, cycloalkyl groups may be substituted with up to 3 substituents which are defined herein by the definition of alkyl.

The term “alkoxy” refers to those hydrocarbon groups having an oxygen bridge and being in either a straight or branched configuration and if two or more carbon atoms in length, they may include a double or a triple bond. Exemplary of such alkoxy groups are methoxy, ethoxy, propoxy, isopropoxy, butoxy, isobutoxy, tertiary butoxy, pentoxy, isopentoxy, hexoxy, isohexoxy allyloxy, propargyloxy, and the like.

“Halogen” or “halo” as used herein means fluoro, chloro, bromo and iodo.

The term “alkenyl” refers to a hydrocarbon radical straight, branched or cyclic containing from 2 to 10 carbon atoms and at least one carbon to carbon double bond. Preferred alkenyl groups include ethenyl, propenyl, butenyl and cyclohexenyl. Preferably, alkenyl is C_{2-C}₆ alkenyl.

Preferably, alkynyl is C₂-C₆ alkynyl.

As used herein, “aryl” is intended to mean any stable monocyclic or bicyclic carbon ring of up to 7 members in each ring, wherein at least one ring is aromatic. Examples of such aryl elements include phenyl, naphthyl, tetrahydronaphthyl, indanyl, biphenyl, phenanthryl, anthryl or acenaphthyl.

The term heterocyclyl, heterocycle or heterocyclic, as used herein, represents a stable 5- to 7-membered monocyclic or stable 8- to 11-membered bicyclic heterocyclic ring which is either saturated or unsaturated, and which consists of carbon atoms and from one to four heteroatoms selected from the group consisting of N, O, and S, and including any bicyclic group in which any of the above-defined heterocyclic rings is fused to a benzene ring. The heterocyclic ring may be attached at any heteroatom or carbon atom which results in the creation of a stable structure. The term heterocyclyl, heterocycle or heterocyclic includes heteroaryl moieties. Examples of such heterocyclic elements include, but are not limited to, azepinyl, benzimidazolyl, benzisoxazolyl, benzofurazanyl, benzopyranyl, benzothiopyranyl, benzofuryl, benzothiazolyl, benzothienyl, benzoxazolyl, chromanyl, cinnolinyl, dihydrobenzofuryl, dihydrobenzothienyl, dihydrobenzothiopyranyl, dihydrobenzothiopyranyl sulfone, 1,3-dioxolanyl, furyl, imidazolidinyl, imidazolinyl, imidazolyl, indolinyl, indolyl, isochromanyl, isoindolinyl, isoquinolinyl, isothiazolidinyl, isothiazolyl, isothiazolidinyl, morpholinyl, naphthyridinyl, oxadiazolyl, 2-oxoazepinyl, oxazolyl, 2-oxopiperazinyl, 2-oxopiperdinyl, 2-oxopyrrolidinyl, piperidyl, piperazinyl, pyridyl, pyrazinyl, pyrazolidinyl, pyrazolyl, pyridazinyl, pyrimidinyl, pyrrolidinyl, pyrrolyl, quinazolinyl, quinolinyl, quinoxalinyl, tetrahydrofuryl, tetrahydroisoquinolinyl, tetrahydroquinolinyl, thiamorpholinyl, thiamorpholinyl sulfoxide, thiazolyl, thiazolinyl, thienofuryl, thienothienyl, and thienyl. An embodiment of the examples of such heterocyclic elements include, but are not limited to, azepinyl, benzimidazolyl, benzisoxazolyl, benzofurazanyl, benzopyranyl, benzothiopyranyl, benzofuryl, benzothiazolyl, benzothienyl, benzoxazolyl, chromanyl, cinnolinyl, dihydrobenzofuryl, dihydrobenzothienyl, dihydrobenzothiopyranyl, dihydrobenzothiopyranyl sulfone, furyl, imidazolidinyl, imidazolinyl, imidazolyl, indolinyl, indolyl, isochromanyl, isoindolinyl, isoquinolinyl, isothiazolidinyl, isothiazolyl, isothiazolidinyl, morpholinyl, naphthyridinyl, oxadiazolyl, 2-oxoazepinyl, oxazolyl, 2-oxopiperazinyl, 2-oxopiperdinyl, 2-oxopyrrolidinyl, piperidyl, piperazinyl, pyridyl, 2-pyridinonyl, pyrazinyl, pyrazolidinyl, pyrazolyl, pyridazinyl, pyrimidinyl, pyrrolidinyl, pyrrolyl, quinazolinyl, quinolinyl, quinoxalinyl, tetrahydrofuryl, tetrahydroisoquinolinyl, tetrahydroquinolinyl, thiamorpholinyl, thiamorpholinyl sulfoxide, thiazolyl, thiazolinyl, thienofuryl, thienothienyl, thienyl and triazolyl.

Preferably, heterocycle is selected from 2-azepinonyl, benzimidazolyl, 2-diazapinonyl, imidazolyl, 2-imidazolidinonyl, indolyl, isoquinolinyl, morpholinyl, piperidyl, piperazinyl, pyridyl, pyrrolidinyl, 2-piperidinonyl, 2-pyrimidinonyl, 2-pyrollidinonyl, quinolinyl, tetrahydrofuryl, tetrahydroisoquinolinyl, and thienyl.

As used herein, “heteroaryl” is intended to mean any stable monocyclic or bicyclic carbon ring of up to 7 members in each ring, wherein at least one ring is aromatic and wherein from one to four carbon atoms are replaced by heteroatoms selected from the group consisting of N, O,and S. Examples of such heterocyclic elements include, but are not limited to, benzimidazolyl, benzisoxazolyl, benzofurazanyl, benzopyranyl, benzothiopyranyl, benzofuryl, benzothiazolyl, benzothienyl, benzoxazolyl, chromanyl, cinnolinyl, dihydrobenzofuryl, dihydrobenzothienyl, dihydrobenzothiopyranyl, dihydrobenzothiopyranyl sulfone, furyl, imidazolyl, indolinyl, indolyl, isochromanyl, isoindolinyl, isoquinolinyl, isothiazolyl, naphthyridinyl, oxadiazolyl, pyridyl, pyrazinyl, pyrazolyl, pyridazinyl, pyrimidinyl, pyrrolyl, quinazolinyl, quinolinyl, quinoxalinyl, tetrahydroisoquinolinyl, tetrahydroquinolinyl, thiazolyl, thienofuryl, thienothienyl, thienyl and triazolyl.

As used herein, unless otherwise specifically defined, substituted alkyl, substituted cycloalkyl, substituted aroyl, substituted aryl, substituted heteroaroyl, substituted heteroaryl, substituted arylsulfonyl, substituted heteroaryl-sulfonyl and substituted heterocycle include moieties containing from 1 to 4 substituents, preferably 1 to 3 substituents in addition to the point of attachment to the rest of the compound. Preferably, such substituents are selected from the group which includes but is not limited to F, Cl, Br, CF₃, NH₂, N(C₁-C₆ alkyl)₂, NO₂, CN, (C₁-C₆ alkyl)O—, (aryl)O—, (C₁-C₆ (C₁-C₆ alkyl)C(O)N—, H₂N—C(NH)—, (C₁-C₆ alkyl)C(O)—, (C₁-C₆ alkyl)OC(O)—, (C₁-C₆ alkyl)OC(O)NH—, phenyl, pyridyl, imidazolyl, oxazolyl, isoxazolyl, thiazolyl, thienyl, furyl, isothiazolyl and C₁-C₂₀ alkyl, (CH₂)ₙOH, CF_{3,} (CH₂)ₙC(O)OH, (CH₂)ₙC(O)OC₁₋₆ alkyl, (CH₂)ₙC(O)NR₇R_{8,} (CH₂)ₙC₅₋₁₀ heterocyclyl, SO₂NR₅R_{6,} (CH₂)C₆₋₁₀ aryl, N(R)_{2,} NO_{2,} CN, (C₁₋₆ alkyl)O—, (aryl)O—, (C₁₋₆ alkyl)S(O)₀₋₂—, C₁₋₁₂ alkyl, said heterocyclyl, and aryl optionally substituted with 1 to 3 groups selected from the group consisting of (CH₂)ₙOR, (CH₂)ₙN(R)_{2,}—O—.

When a functional group is termed “protected”, this means that the group is in modified form to preclude undesired side reactions at the protected site. Suitable protecting groups for the compounds of the present invention will be recognized from the present 30 application taking into account the level of skill in the art, and with reference to standard textbooks, such as Greene, T. W. et al. Protective Groups in Organic Synthesis Wiley, New York (1991). Examples of suitable protecting groups are contained throughout the specification.

The compounds of the present invention are basic, and therefore salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Particularly preferred are citric, hydrobromic, hydrochloric, maleic, phosphoric, sulfuric and tartaric acids.

The preparation of the pharmaceutically acceptable salts described above and other typical pharmaceutically acceptable salts is more fully described by Berg et al., “Pharmaceutical Salts,” J. Pharm. Sci., 1977:66:1-19.

An embodiment of this invention is realized when R₁ represents —C₁₋₆ alkyl, preferably methyl, and C₃₋₆ cycloalkyl, and all other variables are as described herein.

Another embodiment of this invention is realized when R₁ represents H, and all other variables are as described herein.

Another embodiment of this invention is realized when R₂ represents OC₁₋₆ alkyl, preferably the alkyl is methyl, and all other variables are as described herein.

Another embodiment of this invention is realized when R₂ represents OH and all other variables are as described herein.

Another embodiment of this invention is realized when R₂ represents H and all other variables are as described herein.

Another embodiment of this invention is realized when R₅ and R₆ independently represent hydrogen, C₁₋₁₂ alkyl, —(CH₂)ₙC₅₋₁₀ heterocyclyl, —(CH₂)ₙNR₇R_{8,} —(CH₂)ₙNR(CH₂)ₙNR₇R_{8,}—(CH₂)ₙNR(CH₂)ₙC₅₋₁₀ heterocyclyl, —(CH₂)ₙC₅₋₁₀ aryl, —(CHR)ₙNHC(O)(CH₂)ₙNR₇R_{8,}—(CH₂)ₙCHR₇CF_{3,}—C(O)C₁₋₆ alkyl, —C(O)CF_{3,} —C(O)(C(R)₂)ₙNR₁ R_{7,}—C(O)NR(CH₂)ₙC₅₋₁₀ heterocyclyl, —C(O)CHR₅(CH₂)ₙC(O)NR₁ R₁, —C(O)C(O)NR₁R₁, or —C(O)(CH₂)ₙC₅₋₁₀ heterocyclyl, said aryl, and heterocyclyl optionally substituted with one or more groups of R^{a}; said alkyl optionally substituted with 1 to 6 hydroxy and/or optionally substituted by one or more groups of R^{a}.

Another embodiment of this invention is realized when R₅ and R₆ independently represent hydrogen, C₁₋₁₂ alkyl, —(CH₂)ₙC₅₋₁₀ heterocyclyl, —(CH₂)ₙNR₇R_{8,} —(CH₂)ₙNR(CH₂)ₙNR₇R₈, —(CH₂)ₙNR(CH₂)ₙC₅₋₁₀ aryl, —(CH₂)ₙNHC(O)(CH₂)ₙNR₇R_{8,} —C(O)C(R₁ )₂NR₁R_{7,}—C(O)NR(CH₂)ₙC₅₋₁₀ heterocyclyl, —C(O)(CH₂)ₙC₅₋₁₀ heterocyclyl, —C(O)CHR₅(CH₂)ₙC(O)NR₁R₁, or —C(O)(CH₂)ₙC₅₋₁₀ heterocyclyl, said aryl, and heterocyclyl optionally substituted with one or more groups of R^{a}; said alkyl optionally substituted with 1 to 6 hydroxy and/or optionally substituted by one or more groups of R^{a}.

A sub-embodiment of this invention is realized when one of R₅ and R₆ is hydrogen or C₁₋₆ alkyl and the other is hydrogen, C₁₋₁₂ alkyl, —(CH₂)ₙC₅₋₁₀ heterocyclyl, —(CH₂)ₙNR₇R₈, —(CH₂)ₙNR(CH₂)ₙNR₇R_{8,}—(CH₂)ₙNHC(O)(CH₂)ₙNR₇R_{8,} —C(O)C(R₁)₂NR₁R₇, —C(O)NR(CH₂)ₙC₅₋₁₀ heterocyclyl, —C(O)CHR₅(CH₂)ₙC(O)NR₁ R_{1,} or —C(O)(CH₂)ₙC₅₋₁₀ heterocyclyl.

Another embodiment of this invention is realized when R₄ represents

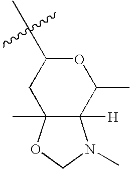

and all other variables are as described herein.

Another embodiment of this invention is realized when R₄ represents

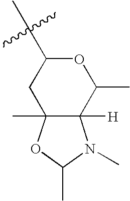

and all other variables are as described herein.

Another embodiment of this invention is realized when R₄ represents

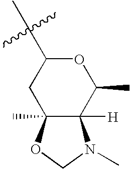

and all other variables are as described herein.

Another embodiment of this invention is realized when R₄ represents

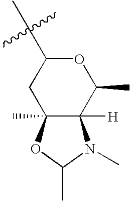

and all other variables are as described herein.

Another embodiment of this invention is realized when R₄ represents

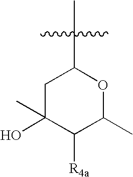

and all other variables are as described herein. A subembodiment of this invention is realized when R₄ₐ is —N(CH₃)₂, —NH_{2,}—NHCH₃, —N+(CH₃)₂O—.

Another embodiment of this invention is realized when R₄ represents

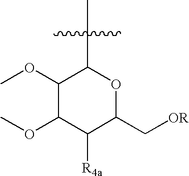

and all other variables are as described herein. A subembodiment of this invention is realized when R₄ₐ is hydrogen and R is hydrogen.

Another embodiment of this invention is realized when R₄ represents

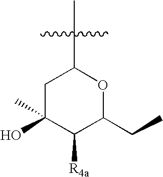

and all other variables are as described herein. A subembodiment of this invention is realized when R₄ₐ is —N(CH₃)₂, —NH₂, —NHCH₃, —N+(CH₃)₂O—.

Another embodiment of this invention is realized when R₄ represents

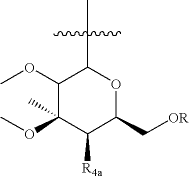

and all other variables are as described herein. A subembodiment of this invention is realized when R₄ₐ is hydrogen and R is hydrogen.

Another embodiment of this invention is realized when R₇ and R₈ are independently selected from the group consisting of hydrogen, C₁₋₆ alkyl (said alkyl group optionally substituted with 1 to 6 groups of C₁₋₄ alkoxy or OH), —(CH₂)ₙN(R)₂, —(CH₂)ₙX (wherein X represents phenyl, pyrimidinyl, morpholinyl, piperazinyl, pridinyl, pyrazolyl, indolyl, furanyl, isoindazolyl, pyrazinyl, pyrrolyl, imidazolyl, triazolyl or teterazolyl said X groups optionally substituted with 1 to 3 groups of R^{a}), Still another embodiment of this invention is realized by structural formula II:

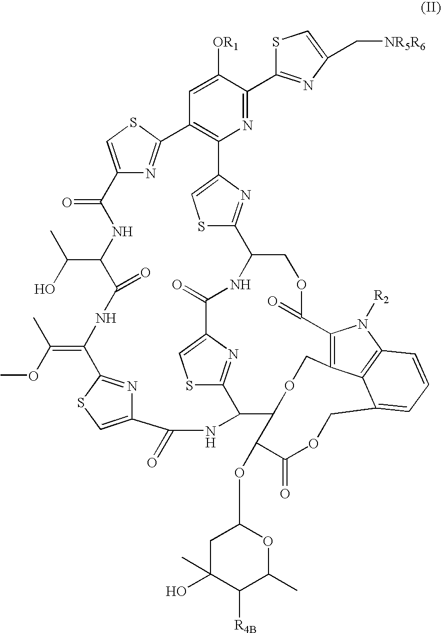

and all other variables are as described herein.

Preferred compounds of this invention are selected from the group of compounds found in Table 1 below:

TABLE 1

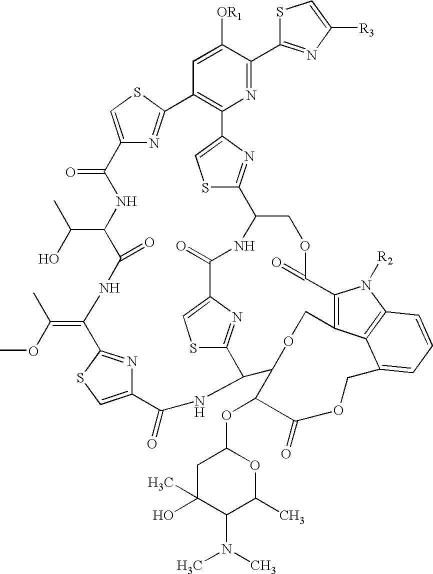

| Compound | R₁ | R₂ | R₃ |
|---|---|---|---|
| 1 | H | OH | 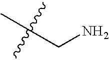 |
| 2 | H | OH | 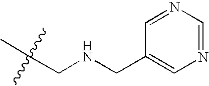 |
| 3 | H | OH | 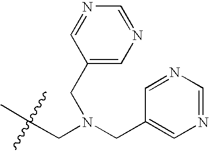 |
| 4 | H | OH | 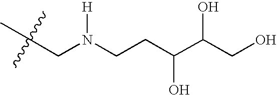 |
| 5 | H | OH | 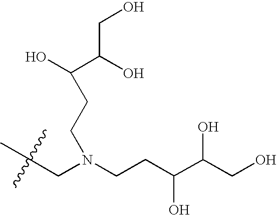 |
| 6 | H | OH | 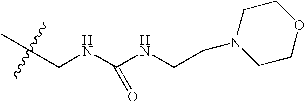 |
| 7 | H | 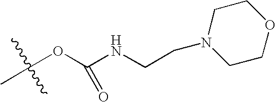 | 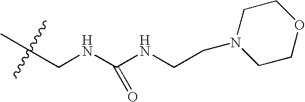 |
| 8 | H | OH | 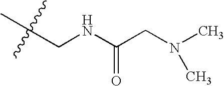 |
| 9 | H | OH | C(O)H |
| 10 | H | OH | 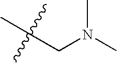 |
| 11 | H | H | 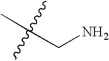 |
| 12 | 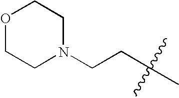 | —CH₂CH₃ | 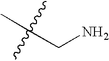 |
| 13 | CH₃ | OH | 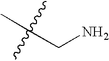 |
| 14 | H | OCH₃ | 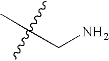 |
| 15 | H | OH | 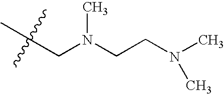 |
| 16 | H | OH | 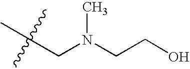 |
| 17 | H | OH | 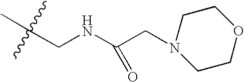 |
| 18 | H | OH | 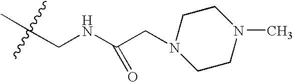 |
| 19 | H | OH | 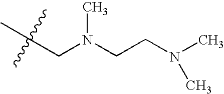 |
| 20 | H | OH | 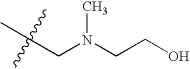 |
| 21 | H | OCH₃ | 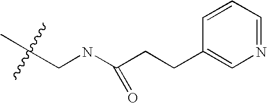 |
| 22 | H | OH | 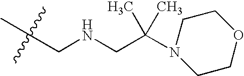 |
| 23 | H | H | 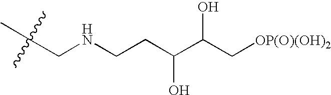 |
| 24 | H | OH | 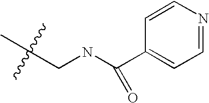 |
| 25 | H | OH | 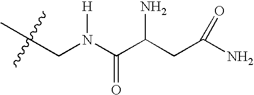 |
| 26 | H | OH | 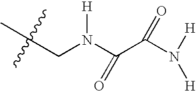 |
| 27 | 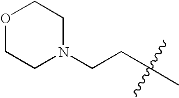 | H | 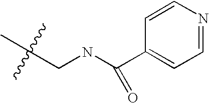 |
| 28 | 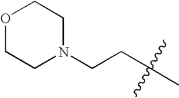 | H | 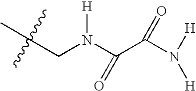 |
| 29 | H | OH | 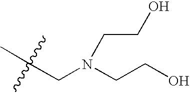 |
| 30 | H | OH | 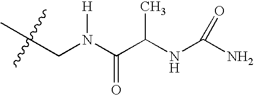 |
| 31 | H | OH | 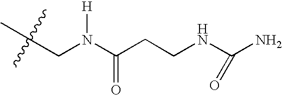 |
| 32 | H | OH | 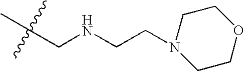 |
| 33 | H | H | 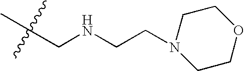 |
| 34 | H | OH | 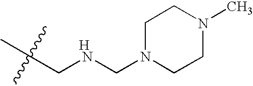 |
| 35 | H | OH | 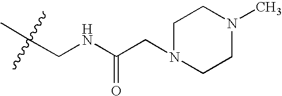 |
| 36 | H | H | 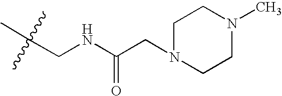 |
| 37 | H | OH | 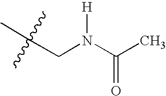 |
| 38 | H | OH | 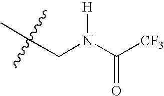 |
| 39 | H | OH | 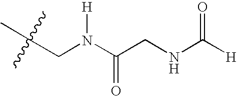 |
| 40 | H | OH | 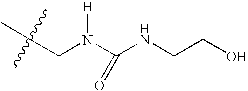 |
| 41 | H | OH | 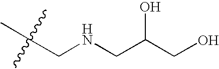 |
| 42 | H | OH | 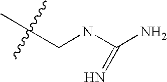 |
| 43 | H | OH | 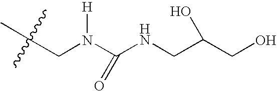 |
| 44 | 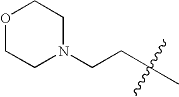 | OH | 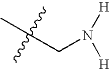 |
| 45 | 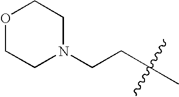 | OH | 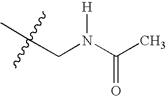 |
| 46 | H | OH | 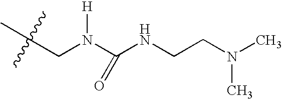 |
| 47 | H | OH | 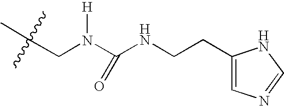 |
| 48 | H | OH | 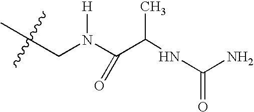 |
| 49 | H | OH | 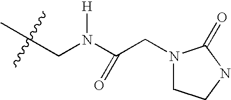 |
| 50 | H | OH | 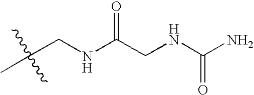 |

and pharmaceutically acceptable salts, esters, enantiomers, diastereomers and mixtures thereof.

The compounds of this invention are a broad spectrum antibiotic useful in the treatment of bacterial infections. They demonstrate antibacterial activity primarily against S. aureus, E. faecalis, E. faecium, S. pneumonieae, B. subtilus including species that are resistant to many known antibiotics. The minimum inhibitory concentration (MIC) values range from 0.0001 to less than 200 μg/mL for test strains such as Staphylococuus aureus, Staphylococuus hemolyticus, Streptococcus pyogenes, Streptococcus pneumoniae, and E. feacalis. The compounds of the invention can be formulated in pharmaceutical compositions by combining the compounds with a pharmaceutically acceptable carrier. Examples of such carriers are set forth below.

The compounds may be employed in powder or crystalline form, in liquid solution, or in suspension. They may be administered by a variety of means; those of principal interest include: topically, orally and parenterally by injection (intravenously or intramuscularly).

Compositions for injection, one route of delivery, may be prepared in unit dosage form in ampules, or in multidose containers. The injectable compositions may take such forms as suspensions, solutions, or emulsions in oily or aqueous vehicles, and may contain various formulating agents. Alternatively, the active ingredient may be in powder (lyophilized or non-lyophilized) form for reconstitution at the time of delivery with a suitable vehicle, such as sterile water. In injectable compositions, the carrier is typically comprised of sterile water, saline or another injectable liquid, e.g., peanut oil for intramuscular injections. Also, various buffering agents, preservatives and the like can be included.

Topical applications may be formulated in carriers such as hydrophobic or hydrophilic bases to form ointments, creams, lotions, in aqueous, oleaginous or alcoholic liquids to form paints or in dry diluents to form powders.

Oral compositions may take such forms as tablets, capsules, oral suspensions and oral solutions. The oral compositions may utilize carriers such as conventional formulating agents, and may include sustained release properties as well as rapid delivery forms.

The dosage to be administered depends to a large extent upon the condition and size of the subject being treated, the route and frequency of administration, the sensitivity of the pathogen to the Compound, the virulence of the infection and other factors. Such matters, however, are left to the routine discretion of the physician according to principles of treatment well known in the antibacterial arts.

The compositions for administration to humans per unit dosage, whether liquid or solid, may contain from about 0.01% to as high as about 99% of Compound I, one embodiment of the range being from about 10-60%. The composition will generally contain from about 15 mg to about 2.5 g of Compound I, one embodiment of this range being from about 250 mg to 1000 mg. In parenteral administration, the unit dosage will typically include pure Compound I in sterile water solution or in the form of a soluble powder intended for solution, which can be adjusted to neutral pH and isotonicity.

The invention described herein also includes a method of treating a bacterial infection in a mammal in need of such treatment comprising the administration of the compound of formula I to the mammal in an amount effective to treat the infection.

One embodiment of the methods of administration of a compound of formula I includes oral and parenteral methods, e.g., i.v. infusion, i.v. bolus and i.m. injection.

For adults, about 5-50 mg of a compound of formula I per kg of body weight given one to four times daily is preferred. The preferred dosage is 250 mg to 1000 mg of the antibacterial given one to four times per day. More specifically, for mild infections a dose of about 250 mg two or three times daily is recommended. For moderate infections against highly susceptible gram positive organisms a dose of about 500 mg three or four times daily is recommended. For severe, life-threatening infections against organisms at the upper limits of sensitivity to the antibiotic, a dose of about 1000-2000 mg three to four times daily may be recommended.

For children, a dose of about 5-25 mg/kg of body weight given 2, 3, or 4 times per day is preferred; a dose of 10 mg/kg is typically recommended.

The compounds of the present invention can be prepared according to Scheme 1, using appropriate materials, and are further exemplified by the following specific examples. The compounds illustrated in the examples are not, however, to be construed as forming the only genus that is considered as the invention. The following examples further illustrate details for the preparation of compounds of the present invention. Those skilled in the art will readily understand that known variations of the conditions and processes of the following preparative procedures can be used to prepare the compounds of the present invention. All temperatures are in degrees Celsius unless otherwise noted.

Compounds of the present invention can be prepared according to Schemes 1-3, using appropriate materials, and are further exemplified by the following specific examples. Thus, starting from the primary amide (isolated from natural source or prepared by degradation from thiazolyl peptide bearing dehydroalanine), the intermediate nitrile is prepared by treatment with TFAA in pyridine. The nitrile is then reduced by catalytic hydrogenation to produce a mixture of the primary amine and the aldehyde in various ratios under different conditions (Scheme 1). Further derivatization of the amine was achieved via various types of reactions (Scheme 2). The aldehyde can be transformed to compounds of Formula II by reductive amination reactions (Scheme 3).

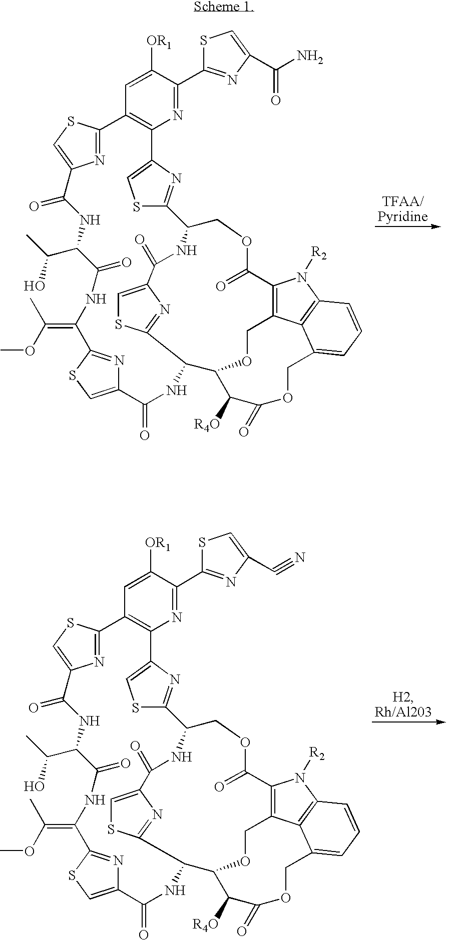

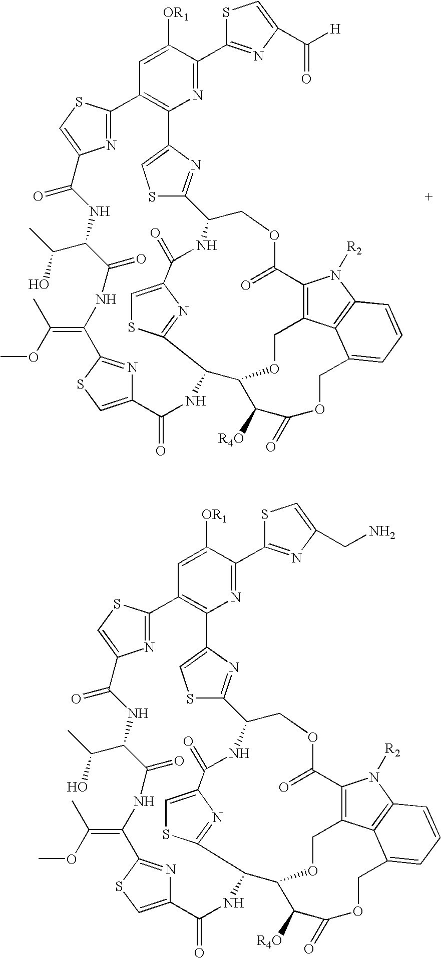

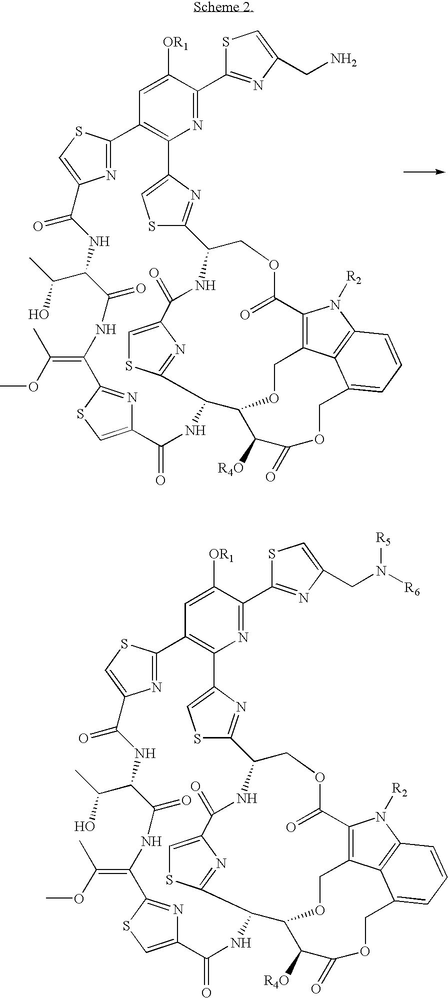

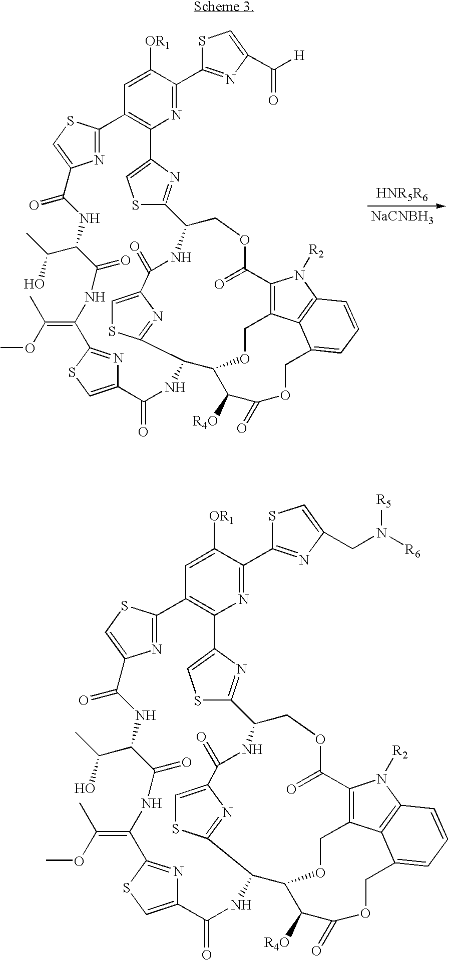

### EXAMPLE 1

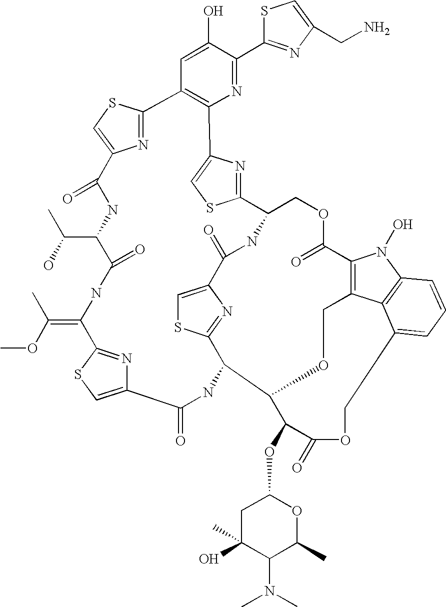

To a suspension of nocathiacin-IV (Prepared according to Regueiro-Ren and Ueda, J. Org. Chem. 2002, 67, 8699) (1 g, 0.73 mmol) in THF (30 mL) at 0° C., was added pyridine (0.59 mL, 7.3 mmol) and trifluoroacetic anhydride (0.52 mL, 3.7 mmol). The resulting solution was stirred at room temperature for 1 h. Volatiles were evaporated, and the residue was purified by silica gel chromatography with 0-10% methanol/dichloromethane. The intermediate nitrile was obtained as the trifluoroacetylated form and as a yellow solid after evaporation (0.5 g, 50% yield). A solution of the nitrile intermediate (0.17 g, 0.12 mmol) in anhydrous methanol containing acetic acid (0.014 mL, 0.24 mmol) was hydrogenated at 50 psi for 24 h with 5% Rhodium on alumina 5 as the catalyst. After filtering off the catalyst and evaporating off the filtrate and washings, the residue was purified by reversed-phase HPLC (Zorbax C-18, 10-70% acetonitrile-water containing 0.1% TFA). The primary amine product was obtained as a yellow solid after lyophilization (TFA salt, 50 mg, 30% yield). NMR ¹H NMR δ (ppm)(CD₃OD): 8.60 (1 H, d, J=9.3 Hz), 8.55 (1 H, s), 8.40 (1 H, s), 8.16 (1 H, s), 8.12 (1 H, d, J=10.8 Hz), 7.87 (1 H, s), 7.83 (1 H, s), 7.81 (1 H, d, J=10.9 Hz), 7.77 (1 H, s), 7.41 (1 H, t, J=7.7 Hz), 7.21 (1 H, d, J=7.2 Hz), 6.13(1 H, d, J=12.3 Hz), 5.88 (1 H, d, J=9.4 Hz), 5.75 (1 H, dd, J=5.0, 11.0 Hz), 5.36 (1 H, dd, J=5.2, 11.3Hz), 5.18(1 H, m), 5.04(1 H, d, J=12.7Hz), 4.95(1 H, d, J=10.7Hz), 4.57 (1 H, d, J=11.2 Hz), 4.39 (1 H, d, J=9.7 Hz), 4.36 (2 H, s), 4.35 (1 H, d, J=4.3 Hz), 4.29 (1 H, d, J=10.7 Hz), 4.11 (1 H, m), 4.05 (1 H, d, J=9.7 Hz), 3.93 (3 H, s), 2.99 (6 H, s), 2.77 (1 H, m), 2.14 (2 H, s), 2.03 (3 H, s), 1.72 (3 H, s), 1.39 (3 H, d, J=6.5 Hz), 0.96 (3 H, s). MS: 1354.26 (M+H), 677.79 [(M+2H)/2].

### EXAMPLE 2

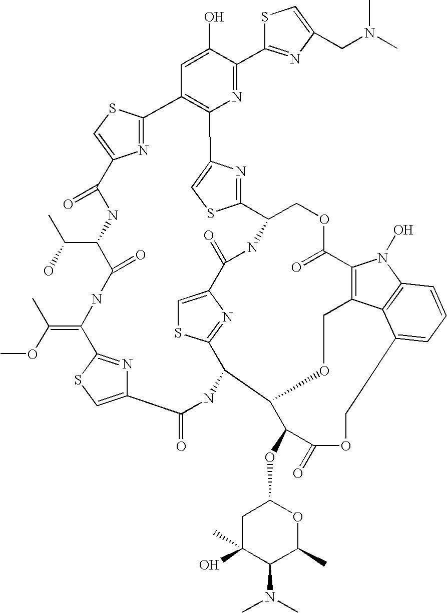

To a solution of the product of example 1 (7.7 mg, 0.006 mmol) in methanol, was added formaldehyde (5 mg, 0.06 mmol), sodium cyanoborohydride (4 mg, 0.06 mmol) and a drop of acetic acid. The mixture was stirred at room temperature for 1 h and quenched with water. Purification with reversed-phase HPLC (10-60% acetonitrile-water with 0.1% TFA) gave product as light yellow solid (TFA salt, 6.1 mg, 80% yield). ¹H NMR δ (ppm)(CD₃OD): 8.64 (1 H, d, J=9.4Hz), 8.59(1 H, s), 8.44(1 H, s), 8.20(1 H, s), 8.16(1 H, d, J=11.0Hz), 8.00(1 H, s), 7.91 (1 H, s), 7.88 (1 H, d, J=6.7 Hz), 7.84 (1 H, d, J=8.4 Hz), 7.82 (1 H, s), 7.44 (1 H, t, J=7.7 Hz), 7.25 (1 H, d, J=7.0 Hz), 6.16 (1 H, d, J=12.4 Hz), 5.92 (1 H, dd, J=1.8, 9.3 Hz), 5.79 (1 H, dd, J=4.9, 10.9 Hz), 5.40 (1 H, dd, J=5.0, 11.4 Hz), 5.23 (1 H, m), 5.08 (1 H, d, J=12.6 Hz), 4.98 (2 H, d, J=10.5 Hz), 4.61 (1 H, d, J=11.3 Hz), 4.58 (2 H, s), 4.43 (1 H, d, J=9.7 Hz), 4.38 (1 H, m), 4.32 (1 H, d, J=10.6 Hz), 4.16 (1 H, q, J=6.6 Hz), 4.08 (1 H, dd, J=1.9, 9.7 Hz), 3.97 (3 H, s), 3.19 (1 H, m), 3.03 (6 H, s), 2.97 (6 H, s), 2.81 (1 H, m), 2.17 (2 H, s), 2.06(3 H, d, J=6.8Hz), 1.76(3 H, s), 1.43 (3 H, d, J=6.1 Hz), 1.01 (3 H, d, J=7.1 Hz). MS: 1382.99 (M+H), 692.37 [(M+2H)/2].

### EXAMPLE 3

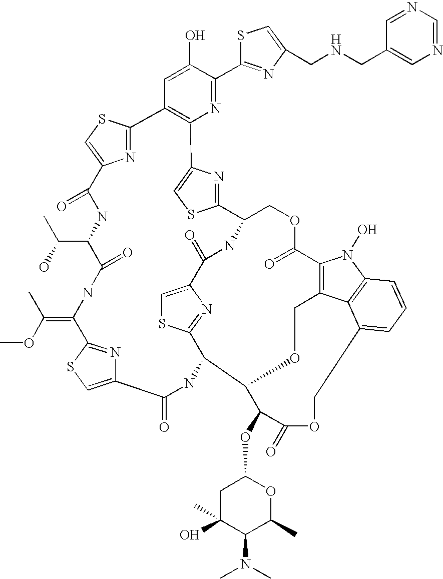

Following the procedure described for example 2 except using pyrimidine-5-carboxaldehyde as the aldehyde component to afford product as a yellow lyophilized solid (TFA salt). ¹H NMR δ (ppm)(CD₃OD): 9.22 (1 H, s), 8.93 (2 H, s), 8.60 (1 H, d, J=9.4 Hz), 8.55 (1 H, s), 8.40 (1 H, s), 8.16 (1 H, s), 8.11 (1 H, d, J=10.7 Hz), 7.91 (1 H, s), 7.86 (1 H, s), 7.84 (1 H, d, J=5.7 Hz), 7.80 (1 H, d, J=8.1 Hz), 7.77 (1 H, s), 7.40 (1 H, t, J=7.5 Hz), 7.20 (1 H, d, J=6.7 Hz), 6.12 (1 H, d, J=12.5 Hz), 5.88 (1 H, dd, J=1.8, .9.6 Hz), 5.75 (1 H, dd, J=4.8, 11.2 Hz), 5.36 (1 H, dd, J=5.1, 11.3Hz), 5.19(1 H, m), 5.04(1 H, d, J=12.7Hz), 4.95(1 H, d, J=10.5Hz), 4.57(3 H, s), 4.44 (2 H, s), 4.39 (1 H, d, J=9.7 Hz), 4.35 (1 H, m), 4.28 (1 H, d, J=10.7 Hz), 4.11 (1 H, q, J=6.9 Hz), 4.04 (1 H, dd, J=1.8, 9.7 Hz), 3.93 (3 H, s), 3.18 (1 H, s), 3.00 (6 H, s), 2.77 (1 H, m), 2.13 (2 H, s), 2.02(3 H, s), 1.73 (3 H, s), 1.39(3 H, d, J=6.1 Hz), 0.97(3 H, d, J=7.1 Hz). MS: 1446.77 (M+H), 724.21 [(M+2H)/2].

### EXAMPLE 4

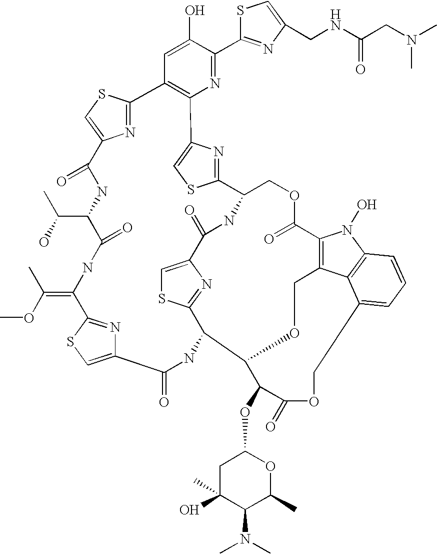

The product of example 1 (10 mg, 0.007 mmol) was mixed with dimethyglycine (2.3 mg, 0.009 mmol), EDC (2.2 mg, 0.011 mmol) and HOBT (1.4 mg, 0.009 mmol) in anhydrous DMF (0.2 mL). The mixture was stirred at room temperature for 1 h, followed by reversed-phase HPLC purification (10-60% acetonitrile with 0.1% TFA). Product was obtained as a yellow lyophilized solid (TFA salt, 3.2 mg, 45% yield). ¹H NMR δ (ppm)(CD₃OD): 8.60 (1 H, d, J=9.4 Hz), 8.54 (1 H, s), 8.40(1 H, s), 8.16(1 H, s), 8.11 (1 H, d, J=11.3Hz), 7.84(1 H, d, J=6.8Hz), 7.82(1 H, s), 7.81 (1 H, d, J=8.4 Hz), 7.76 (1 H, s), 7.58 (1 H, s), 7.41 (1 H, t, J=7.7 Hz), 7.21 (1 H, d, J=6.8Hz),6.12(1 H, d, J=12.5Hz),5.88(1 H, d, J=7.9Hz),5.75(1 H, dd, J=4.7, 11.2 Hz), 5.36(1 H, dd, J=5.1, 11.2Hz), 5.17(1 H, m), 5.04(1 H, d, J=12.7Hz), 4.94(1 H, d, J=10.7 Hz), 4.64 (2 H, s), 4.39 (1 H, d, J=9.7 Hz), 4.35 (1 H, m), 4.28 (1 H, d, J=10.4 Hz), 4.11 (1 H, m), 4.04 (1 H, d, J=11.8 Hz), 3.93 (3 H, s), 3.71 (1 H, m), 2.96 (4 H, s), 2.75 (6 H, s), 2.13 (2 H, s), 2.02 (6 H, d, J=8.1 Hz), 1.71 (3 H, s), 1.39 (3 H, d, J=6.1 Hz), 0.96 (3 H, s). MS: 1442.61 (M+H), 721.88 [(M+2H)/2].

### EXAMPLE 5

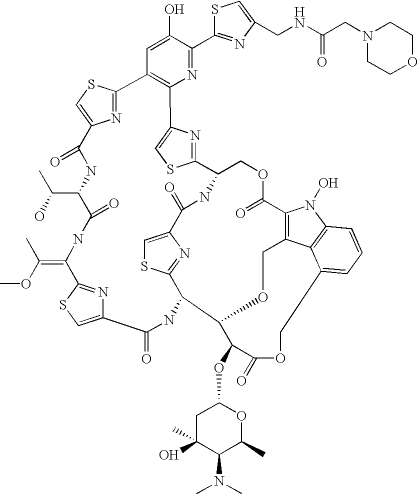

Following the procedure described for example 4 except using 2-(4-morpholinyl) acetic acid as the acid component to afford product as a yellow lyophilized solid (TFA salt). ¹H NMR δ (ppm)(CD₃OD): 8.64 (1 H, d, J=9.3 Hz), 8.56 (1 H, s), 8.44 (1 H, s), 8.20 (1 H, s), 8.15 (1 H, d, J=1.0 Hz), 7.88 (1 H, d, J=6.8 Hz), 7.86 (1 H, s), 7.84 (1 H, s), 7.80 (1 H, s), 7.57 (1 H, s), 7.44 (1 H, t, J=7.7 Hz), 7.30 (2 H, t, J=6.6 Hz), 7.24 (1 H, d, J=6.9 Hz), 6.91 (2 H, dd, J=3.7, 8.6Hz), 6.16(1 H, d, J=12.3Hz), 5.92(1 H, d, J=9.3Hz), 5.79(1 H, dd, J=4.8, 11.0 Hz), 5.40(1 H, dd, J=5.1, 11.3Hz), 5.22(1 H, s), 5.08(2 H, d, J=12.7Hz), 5.02(4 H, d, J=9.2 Hz), 4.98(4 H, d, J=10.6Hz), 4.66(6 H, s), 4.61 (3 H, d, J=11.4Hz), 4.43 (1 H, d, J=9.7 Hz), 4.38 (1 H, m), 4.32 (1 H, d, J=10.5 Hz), 4.16 (1 H, m), 4.08 (1 H, d, J=9.7 Hz), 3.97 (3 H, s), 3.80 (8 H, s), 3.23 (4 H, t, J=6.6 Hz), 3.17 (2 H, t, J=6.7 Hz), 3.04 (7 H, s), 2.97 (3 H, s), 2.81 (2H, s), 2.68(3 H, s), 2.18(2 H, s), 2.07(3 H, s), 1.96(1 H, s), 1.85(1 H, t, J=6.9Hz), 1.76 (3 H, s), 1.69 (1 H, m), 1.59 (1 H, m), 1.42 (3 H, d, J=6.1 Hz), 1.01 (3 H, d, J=7.1 Hz). MS: 1482.21 (M+H), 741.90 [(M+2H)/2].

### EXAMPLE 6

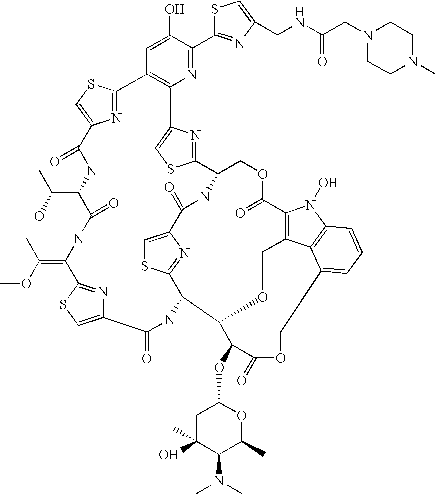

Following the procedure described for example 4 except using 4-methyl-1-piperazine acetic acid as the acid component to afford product as a yellow lyophilized solid (TFA salt). ¹H NMR δ (ppm)(CD₃OD): 8.62 (1 H, d, J=9.3 Hz), 8.57 (1 H, s), 8.42 (1 H, s), 8.18 (1 H, s), 8.14 (1 H, d, J=11.0 Hz), 7.87 (1 H, d, J=6.9 Hz), 7.85 (1 H, s), 7.83 (1 H, d, J=8.4 Hz), 7.78 (1 H, s), 7.58 (1 H, s), 7.43 (1 H, dd, J=7.0, 8.4 Hz), 7.23 (1 H, d, J=7.1 Hz), 6.15 (1 H, d, J=12.4 Hz), 5.91 (1 H, dd, J=1.8, 9.3 Hz), 5.77 (1 H, dd, J=4.7, 10.8 Hz), 5.39 (1 H, dd, J=5.2, 11.4 Hz), 5.21 (1 H, s), 5.07 (1 H, d, J=12.7 Hz), 4.97 (1 H, d, J=10.5 Hz), 4.65 (2 H, s), 4.59 (1 H, d, J=11.2 Hz), 4.42(1 H, d, J=9.7Hz), 4.37(1 H, dd, J=4.4, 6.8Hz), 4.31 (1 H, d, J=10.6 Hz), 4.15 (1 H, m), 4.07 (1 H, dd, J=1.9, 9.7 Hz), 3.96 (3 H, s), 3.27 (2 H, s), 3.20 (1 H, s), 3.02 (7 H, s), 2.90 (3 H, s), 2.80 (1 H, t, J=5.2 Hz), 2.65 (2 H, s), 2.17 (2 H, s), 2.05 (3 H, s), 1.75 (3 H, s), 1.41 (3 H, d, J=6.5 Hz), 0.99 (3 H, d, J=7.0 Hz). MS: 1495.70 (M+H), 748.30 [(M+2H)/2].

### EXAMPLE 7

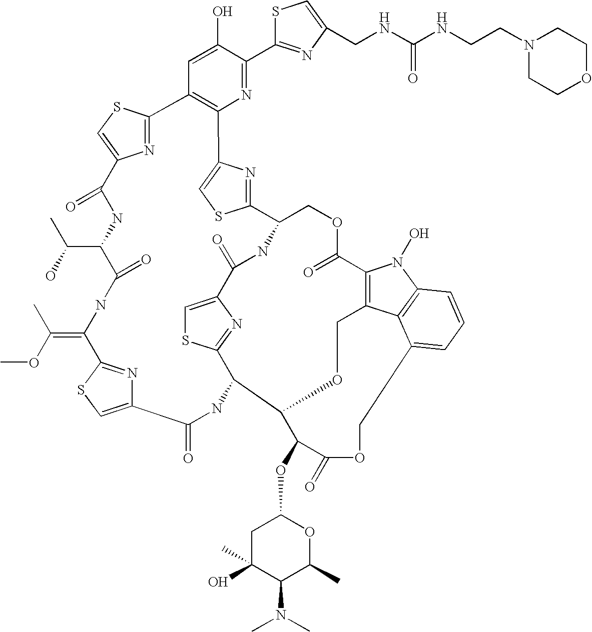

To a solution of 2-(4-aminoethyl) morpholine (0.65 g, 5 mmol) in anhydrous THF at 0° C. was added pyridine (0.42 mL, 5.2 mmol) and 4-nitrophenyl chloroformate (1.3 g, 6.3 mmol) dropwise. The resulting mixture was stirred for 1 h followed by aqueous workup. Purification by silica gel chromatography afforded 4-nitrophenyl-N-(2-morpholinoethyl) carbamate as a yellow solid (0.4 g, 27% yield). The carbamate intermediate (1 mg, 0.004 mmol) was mixed with the product of example 1 (5 mg, 0.004 mmol) and diisopropylethylamine (0.6 μL, 0.004 mmol) in DMSO (0.15 mL) and stirred for 2 h. Reversed-phase HPLC purification afforded product as a lyophilized yellow solid (0.5 mg, 9% yield). ¹H NMR δ (ppm)(CD₃OD): 8.63 (2 H, d, J=9.4 Hz), 8.57 (2 H, s), 8.42 (2 H, s), 8.18 (2 H, s), 8.15 (2 H, d, J=10.9 Hz), 7.86 (2 H, s), 7.83 (2 H, d, J=8.4 Hz), 7.79 (2 H, s), 7.66 (2 H, s), 7.43 (2 H, t, J=7.7 Hz), 7.23 (2 H, d, J=7.0 Hz), 6.15 (2 H, d, J=12.4 Hz), 5.90 (2 H, d, J=9.0 Hz), 5.77 (2 H, dd, J=4.6, 10.7 Hz), 5.38 (2 H, dd, J=5.2, 11.4 Hz), 5.16 (2 H, s), 5.06 (2 H, d, J=12.7 Hz), 4.96 (2 H, d, J=10.6 Hz), 4.65 (4 H, s), 4.59 (5 H, d, J=8.4 Hz), 4.41 (2 H, d, J=9.8 Hz), 4.37 (2 H, d, J=4.3 Hz), 4.31 (2 H, d, J=10.5 Hz), 4.06 (3 H, d, J=10.0 Hz), 3.96 (6 H, s), 2.92 (7 H, s), 2.78 (3 H, s), 2.11(3 H, s), 2.05 (6 H, s), 1.94 (1 H, s), 1.68 (5 H, s), 1.41 (6 H, d, J=6.1 Hz), 0.92 (4 H, s). MS: 1511.70 (M+H), 756.29 [(M+2H)/2].

### EXAMPLE 8

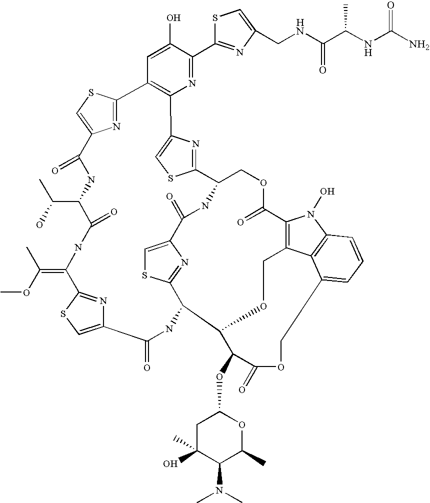

Following the procedure described for example 4 except using N-carbamyl-L-alanine as the acid component to afford product as a yellow lyophilized solid (TFA salt). ¹H NMR δ (ppm) (CD₃OD): 8.67 (1 H, m), 8.62 (1 H, d, J=9.5 Hz), 8.54 (1 H, s), 8.42 (1 H, s), 8.18 (1 H, s), 8.13 (1 H, d, J=10.8 Hz), 7.87 (1 H, d, J=6.6 Hz), 7.83 (2 H, s), 7.79 (1 H, s), 7.56 (1 H, s), 7.43 (1 H, t, J=7.8 Hz), 7.23 (1 H, d, J=6.9 Hz), 6.14 (1 H, d, J=12.8 Hz), 5.91 (1 H, d, J=9.2 Hz), 5.77 (1 H, dd, J=5.2, 10.6 Hz), 5.38 (1 H, dd, J=5.6, 11.4 Hz), 5.21 (1 H, s), 5.06 (1 H, d, J=12.5 Hz), 4.96 (1 H, d, J=10.6 Hz), 4.61 (2 H, d, J=5.4 Hz), 4.58 (1 H, s), 4.41 (1 H, d, J=9.7 Hz), 4.37 (1 H, d, J=4.6 Hz), 4.32-4.24 (2 H, m), 4.15 (1 H, m), 4.06 (1 H, d, J=9.1 Hz), 3.96 (3 H, s), 3.20 (1 H, s), 3.02 (6 H, s), 2.81 (1 H, s), 2.17 (2 H, s), 2.06 (3 H, s), 1.75 (3 H, s), 1.41 (3 H, d, J=5.8Hz), 1.38(3 H, d, J=7.1 Hz), 1.00(3 H, d, J=7.1 Hz). MS: 1469.11 (M+H), 735.07 [(M+2H)/2].

### EXAMPLE 9

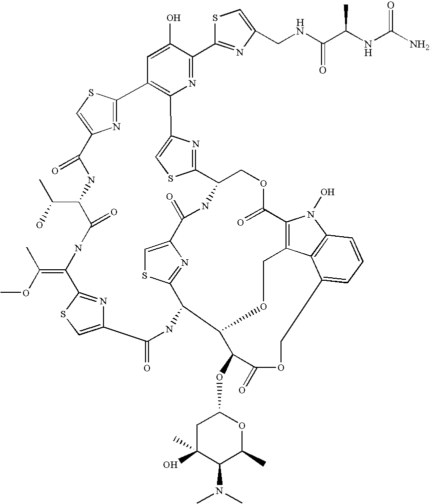

Following the procedure described for example 4 except using N-carbamyl-D-alanine as the acid component to afford product as a yellow lyophilized solid (TFA salt). ¹H NMR δ (ppm) (CD₃OD): 8.62(1 H, d, J=9.12Hz), 8.54(1 H, s), 8.41 (1 H, s), 8.17(1 H, s), 8.15 (1 H, d, J=10.32 Hz), 7.86 (1 H, d, J=7.8 Hz), 7.82 (2 H, d, J=8.1 Hz), 7.78 (1 H, s), 7.56 (1 H, s), 7.42 (1 H, t, J=15.5 Hz), 7.22 (1 H, d, J=6.7 Hz), 6.13 (1 H, d, J=12.8 Hz), 5.88 (1 H, s), 5.76 (1 H, dd, J=4.9, 11.2Hz), 5.37(1 H, dd, J=4.9, 11.4Hz), 5.06(1 H, d, J=12.4Hz), 4.60(2 H, d, J=3.4 Hz), 4.58 (2 H, s) 4.41 (1 H, d, J=9.4 Hz), 4.36 (1 H, m), 4.28 (2 H, dd, J=11.5, 19.8 Hz), 4.05 (2 H, d, J=8.65 Hz), 3.95 (3 H, s), 3.35 (7 H, s), 2.76 (1 H, s), 2.05 (5 H, s), 1.94 (1 H, s), 1.41 (3 H, d, J=6.2 Hz), 1.37 (3 H, d, J=7.2 Hz). MS: 1469.01 (M+H), 735.01 [(M+2H)/2].

### EXAMPLE 10

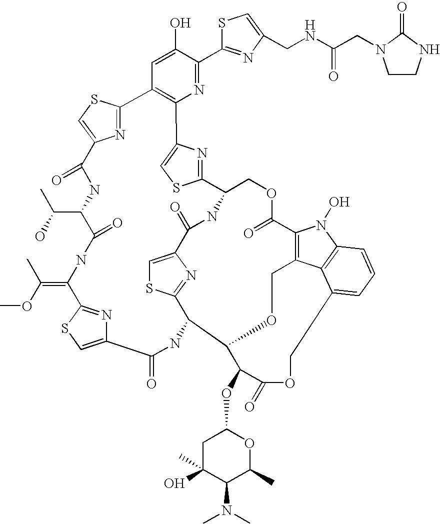

To a solution of 2-imidazolidinone (1 g, 11.6 mmol) in DMF (25 mL) was added cesium carbonate (15.1 g, 46.5 mmol) and t-butyl bromoacetate (2.3 g, 11.6 mmol). The mixture was stirred at room temperature for 2 h. It was partitioned between EtOAc and water. The organic phase was washed with water and brine, dried over Na₂SO₄ and evaporated. Purification by silica gel chromatography afforded the t-butyl 2-oxo-1-imidazolidineacetate which yielded the desired acid upon treatment with TFA. (0.3 g, 10% yield). Following the procedure described for example 4 except using 2-oxo- 1-imidazolidineacetic acid as the acid component to afford product as a yellow lyophilized solid (TFA salt). ¹H NMR δ (ppm) (CD₃OD): 8.64 (1 H, d, J=9.2 Hz), 8.55 (2 H, s), 8.41 (1 H, s), 8.17 (2 H, s), 8.15 (1 H, s), 7.83(1 H, s), 7.82 (2 H, s), 7.78 (2 H, s), 7.56 (2 H, s), 7.42 (2 H, t, J=7.9 Hz), 7.23 (2 H, d, J=6.9 Hz), 6.14 (2 H, d, J=12.4 Hz), 5.87 (2 H, s), 5.76 (2 H, dd, J=5.4, 11.4 Hz), 5.38 (2 H, dd, J=5.5, 11.7 Hz), 5.06 (4 H, d, J=12.6 Hz), 4.94 (4 H, d, J=10.5 Hz), 4.63 (3 H, s), 4.59 (5 H, s), 4.41 (2 H, d, J=9.7 Hz), 4.36 (2 H, d, J=4.4 Hz), 4.30 (2 H, d, J=10.8 Hz), 4.04 (2 H, d, J=9.6 Hz), 3.95 (5 H, s), 3.90 (4 H, s), 3.57 (4 H, t, J=7.8 Hz), 3.47 (5 H, t, J=8.0 Hz), 3.17 (1 H, s), 2.76 (2 H, s), 2.63 (9 H, s), 2.05 (4 H, s), 2.02 (3 H, s), 1.92 (1 H, s), 1.57 (5 H, s), 1.41 (5 H, d, J=6.3 Hz), 0.78 (4 H, d, J=6.2 Hz). MS: 1481.11 (M+H), 741.02 [(M+2H)/2].

### EXAMPLE 11

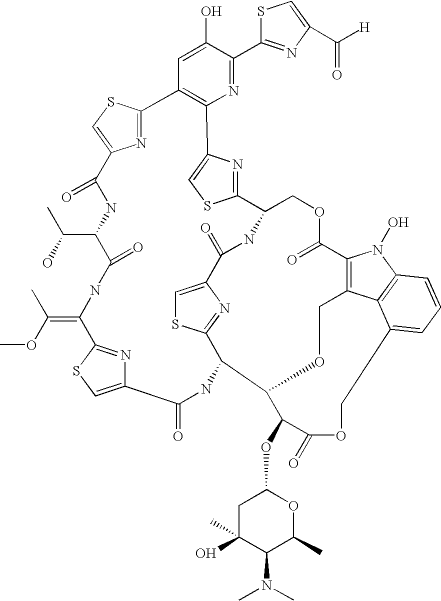

Product was isolated from the same reaction mixture during the preparation of example 1 (14% yield). It was separated from example 1 by reversed-phase HPLC. ¹H NMR δ (ppm)(CD₃OD): 8.83(1 H, s), 8.60 (1 H, d, J=9.4Hz), 8.52 (1 H, s), 8.40 (1 H, s), 8.16 (1 H, s), 8.11 (1 H, d, J=10.8 Hz), 7.85 (1 H, d, J=6.6 Hz), 7.81(2 H, m), 7.69 (1 H, s), 7.40 (1 H, t, J=7.3 Hz), 7.20 (1 H, d, J=7.1 Hz),6.11 (1 H, d, J=12.3Hz),5.88(1 H, d, J=9.2Hz),5.75(1 H, m),5.71 (1 H, s), 5.35 (1 H, m), 5.19 (1 H, s), 5.03 (1 H, d, J=12.5 Hz), 4.94 (1 H, d, J=10.6 Hz), 4.57 (1 H, d, J=11.2Hz), 4.39(1 H, d, J=9.7Hz), 4.33(1 H, m), 4.28 (1 H, d, J=10.6Hz), 4.12(1 H, m), 4.04 (1 H, d, J=9.5 Hz), 3.93 (3 H, s), 3.18 (1 H, s), 3.00 (6 H, d, J=2.6 Hz), 2.78 (1 H, m), 2.14 (2 H, s), 2.03 (3 H, s), 2.02 (5 H, s), 1.91 (1 H, s), 1.73 (3 H, s), 1.39 (3 H, m), 0.97 (3 H, d, J=7.1 Hz). MS: 1353.59 (M+H), 677.61 [(M+2H)/2].

### EXAMPLE 12

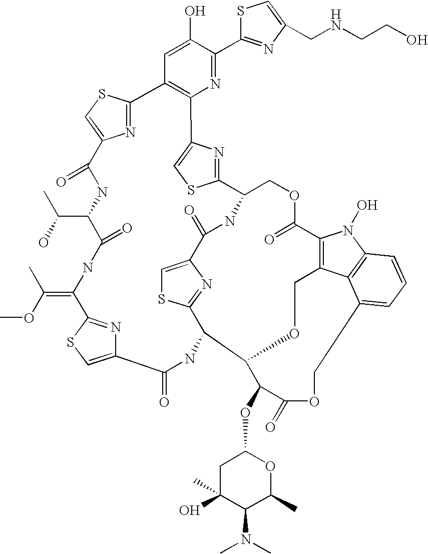

To a solution of the aldehyde of example 11 (1.2 mg, 0.001 mmol) and 2-hydroxyethylamine (2 μL) in methanol was added a drop of acetic acid, the mixture was stirred for 30 minutes before NaBH₃CN (3 mg) was added. After stirring for 30 minutes, the mixture was quenched with water. Purification by reversed-phase HPLC (Xterra C18, 10-50% acetonitrile/water containing 0.1% TFA) afforded product as a yellow solid (1 mg, 80% yield). MS: 1382.99 (M+H), 692.37 [(M+2H)/2]. ¹H NMR δ (ppm)(CD₃OD): 8.62 (1 H, d, J=9.5 Hz), 8.56 (1 H, s), 8.42 (1 H, s), 8.18 (1 H, s), 8.14 (1 H, d, J=10.9 Hz), 7.86 (2 H, s), 7.82 (1 H, d, J=8.2 Hz), 7.79 (1 H, s), 7.43(1 H, t, J=7.7Hz), 7.23 (1 H, d, J=6.9Hz), 6.14(1 H, d, J=12.6Hz), 5.90(1 H, d, J=9.0 Hz), 5.77 (1 H, dd, J=4.9, 10.6 Hz), 5.38 (1 H, dd, J=5.3, 11.6 Hz), 5.17 (1 H, s), 5.06 (1 H, d, J=12.7 Hz), 4.96 (1 H, d, J=10.7 Hz), 4.59 (26 H, s), 4.46 (2 H, s), 4.41 (1 H, d, J=9.8 Hz), 4.36 (1 H, d, J=4.3 Hz), 4.30 (1 H, d, J=10.6 Hz), 4.10 (1 H, m), 4.06 (1 H, d, J=9.5 Hz), 3.95 (3 H, s), 3.85 (2 H, t, J=5.2 Hz), 3.19 (3 H, t, J=5.0 Hz), 2.92 (7 H, s), 2.77 (1 H, s), 2.13 (2 H, s), 2.05 (3 H, s), 1.70 (3 H, s), 1.41 (3 H, d, J=6.1 Hz), 0.95 (3 H, s). MS: 1399.77 (M+H), 700.32 [(M+2H)/2].

### EXAMPLE 13

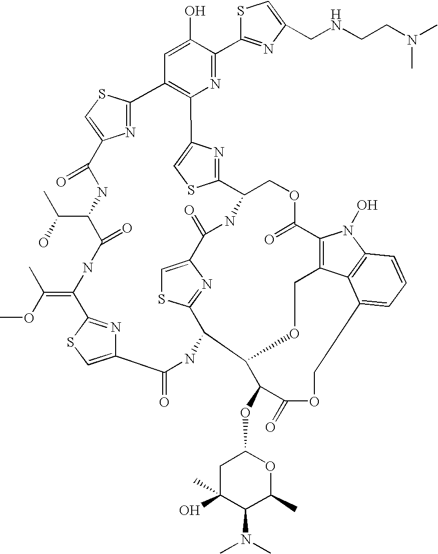

Following the procedure described for example 12 except using N,N-dimethylethylenediamine as the amine component afforded product as a lyophilized yellow solid (TFA salt). ¹H NMR δ (ppm)(CD₃OD): 8.62 (1 H, d, J=9.3 Hz), 8.56 (1 H, s), 8.42 (1 H, s), 8.18 (1 H, s), 8.14 (1 H, d, J=10.9 Hz), 7.87 (1 H, s), 7.85 (1 H, s), 7.83 (1 H, d, J=8.6 Hz), 7.78 (1 H, s), 7.71 (1 H, s), 7.43 (1 H, m), 7.23 (1 H, d, J=6.9 Hz), 6.15 (1 H, d, J=12.4 Hz), 5.90 (1 H, d, J=7.5 Hz), 5.77 (1 H, dd, J=5.6, 11.1 Hz), 5.39 (1 H, dd, J=5.1, 11.3 Hz), 5.21 (1 H, s), 5.06 (2 H, d, J=12.5 Hz), 4.97 (3 H, d, J=10.6 Hz), 4.60 (1 H, d, J=11.3 Hz), 4.41 (1 H, d, J=9.7 Hz), 4.37 (1 H, t, J=5.6Hz), 4.31 (1 H, d, J=10.6Hz), 4.20(2 H, s), 4.15 (1 H, m), 4.07(1 H, d, J=9.9Hz), 3.96 (3 H, s), 3.20 (1 H, s), 3.17 (2 H, s), 3.11 (2 H, s), 3.02 (7 H, s), 2.80 (8 H, s), 2.17 (2 H, s), 2.05 (3 H, s), 1.75 (4 H, s), 1.41 (4 H, d, J=6.2 Hz), 0.99 (4 H, d, J=7.1 Hz). MS: 1426.85 (M+H), 713.85 [(M+2H)/2].

The antibacterial activity of the compounds of Formula I can be determined using the assay methods described below.

### Materials:

Cation-Adjusted Mueller Hinton Broth (MH; BBL)
50% Lysed Horse Blood (LHB; BBL) (stored frozen)
RPMI 1640 (BioWhittaker)
Human Serum (Pel-Freez)
RPMI 1640 (Bio Whittaker)
Haemophilus Test Medium (HTM, Remel)
Trypticase Soy Broth (TSB, 5 mL/tube; BBL)
0.9% Sodium Chloride (Saline; Baxter)
Trypticase Soy+5% Sheep Blood Agar Plates (TSA; BBL)
Sabouraud Dextrose Agar Plates (BBL)
Chocolate Agar Plates (BBL)
2× Skim Milk (Remel)
Microbank Beads (Kramer Scientific)
MIC 2000 Microtiter plate inoculator.
2× Trypticase Soy Broth (TSB, BBL)+15% glycerol/50% horse serum.
96-Well Microtiter plates, lids, inoculum trays (Dynex Laboratories)
8-Channel Finn Multichannel pipettor, 0.5-10 μL volume

### Methods:

Media Preparation

Cation-Adjusted Mueller Hinton Broth (BBL): Prepared according to manufacturer's instructions (22 gms dissolved in 1000 mL water; autoclaved 22 minutes). Stored refrigerated. Filter-sterilized before use using a Coming 0.45 Tm cellulose acetate filter.

50% Lysed Horse Blood: Defibrinated horse blood is diluted 1:1 with sterile distilled water; frozen, thawed and re-frozen (at least 7 times), then centrifuged. Stored frozen at −20° C.

Cation-Adjusted Mueller Hinton+2.5% Lysed Horse Blood: Aseptically add 5 mL 50% lysed horse blood to 100 mL Cation-Adjusted Mueller Hinton Broth. Filter-sterilize before use using a Coming 0.45 Tm cellulose acetate filter.

Cation-Adjusted Mueller Hinton+50% Human Serum: Aseptically add 50 mL Human Serum to 50 mL 2× Cation-Adjusted Mueller Hinton Broth. Filter-sterilize before use using a Corning 0.45 Tm cellulose acetate filter.

Haemophilus Test Medium (Remel): Received prepared from manufacturer. Filter-sterilized before use using a Coming 0.45 Tm cellulose acetate filter.

0.9% Sodium Chloride (Saline; Abbott Labs): Received prepared from manufacturer.

2× Skim Milk (Remel): Received prepared from manufacturer.

All agar plates are received prepared from manufacturer.

| CONDITIONS AND | |
|---|---|
| INOCULUM | FOR REPRESENTATIVE STRAINS |
| BACILLUS, | INCUBATION CONDITIONS, 35° C.; MICS READ AT 18-22 |
| STAPHYLOCOCCUS, | HOURS; |
| ENTEROCOCCUS: | |
| ESCHERICHI:, | CATION-ADJUSTED MUELLER HINTON (CAMHB; BBL); |
| | INOCULUM = 10⁵ CFU/ML |
| STREP. PNEUMONIAE: | INCUBATION CONDITIONS, 35° C.; MICS READ AT 22-24 HOURS; |
| | CATION-ADJUSTED MUELLER HINTON + 2.5% LYSED |
| | HORSE BLOOD (LHB); INOCULUM = 10⁵ CFU/ML |
| HAEMOPHILUS | INCUBATION CONDITIONS, 35° C.; MICS READ AT 18-22 HOURS; |
| INFLUENZAE: | HAEMOPHILUS TEST MEDIUM (HTM; REMEL); INOCULUM = |
| | 10⁵ CFU/ML |
| CANDIDA: | INCUBATION CONDITIONS, 35° C.; MICS READ AT 24 |
| | HOURS; RPMI 1640 MEDIUM (BIOWHITTAKER) |
| | INOCULUM = 10³ CFU/ML |

HIGHEST CONCENTRATION OF ANTIBIOTIC TESTED = 64 μG/ML (WHEN STARTING FROM A 1 MG/ML SOL'N IN 50% DMSO)
FINAL CONCENTRATION OF DMSO PER WELL = 3.2%

### Selection and Maintenance of Isolates

The type of strains listed above can be obtained from publicly available sources. The strain of Haemophilus influenzae used in to assay the compound of this invention is a mouse pathogen used for in vivo testing at Merck. The Escherichia coli strain used in to assay the compound of this invention is a cell wall permeable strain. The Candida albicans strain is used as a control. These culture are maintained as frozen stocks at −80° C. in a) Microbank beads; b) 2× Skim Milk; or c) in 2× X Trypticase Soy Broth+15% glycerol/50% horse serum (Haemophilus and Streptococcus pneumoniae).

### Inoculum Preparation

Selected isolates are sub-cultured onto either Chocolate Agar Plates (Haemophilus influenzae), onto Trypticase Soy+5% Sheep Blood Agar Plates (Streptococcus pneumoniae, Staphylococcus aureus, Escherichia coli, Enterococcus, Bacillus) or onto Sabouraud Dextrose Agar (Candida) and incubated at 35° C. Haemophilus and Streptococcus pneumoniae are incubated in 5% CO₂; all other isolates are incubated in ambient air. Isolates are sub-cultured 2× before assay.

Colonies are selected from plates and used to prepare an inoculum equivalent to a 0.5 McFarland standard in Trypticase Soy Broth. An inoculum with a density equivalent to a 1.0 McFarland standard is prepared for Streptococcus pneumoniae. The inoculum density for all cultures is ˜10⁸ CFU/mL in TSB. This TSB inoculum is diluted 1:10 in sterile saline (4 mL inoculum+36 mL saline; equivalent to ˜10⁷ CFU/mL) and kept on ice until used to inoculate microtiter plates.

Colony counts are performed on randomly-selected isolates to confirm CFU/well (TSB inoculum plated out 10⁻⁵, 10⁻⁶ onto either TSA II+5% SB or onto chocolate agar plates, incubated overnight, 35° C., CO₂)

### Plate Filling

All wells of 96-well microtiter plates (Dynex) are filled with 100 TL media. Haemophilus test media plates are prepared to test Haemophilus influenzae; Cation-Adjusted Mueller Hinton+5% Lysed Horse Blood plates are prepared to test Streptococcus pneumoniae; Cation-Adjusted Mueller Hinton Broth plates are prepared to test Enterococcus, Staphylococcus aureus, Escherichia coli and Bacillus subtilis. RPMI 1640 is used to test Candida. The MICs against S. aureus Smith are determined in Cation-adjusted Mueller Hinton and in Cation-Adjusted Mueller Hinton+50% Human Serum, to determine if the compound is inactivated by some component in serum (indicated by an increase in the MIC). Filled plates are wrapped in plastic bags (to minimize evaporation), stored frozen and thawed before use.

### Preparation of Compounds

The compounds are prepared on a weight basis. Compounds are prepared to 2-10 mg/mL in 100% DMSO, then diluted to 1 mg/mL in a 1:1 dilution of DMSO/233 CAMHB (final concentration=50% DMSO/50% CAMHB). Compounds are serially diluted 1:1 in 50% DMSO/50% CAMHB in BD Biosciences Deep Well Polypropylene 96 well plates (starting concentration 1-5 mg/mL).

### Microbroth Dilution Assay

Using a Finn Automated Multichannel Pipette, (0.5-10 μL volume) 6.4 TLs of antimicrobial working solutions are added to wells of filled microtiter plates (concentration of antimicrobial in first well=512-64 microg/mL; concentration of DMSO=3.2%). Antimicrobials are added in this manner to keep constant the amount of DMSO in each well (to keep compounds solubilized and to account for the possibility of non-specific killing by the DMSO. The last row contains a growth control of 3.2% DMSO.

Controls (Penicillin G and chloramphenicol) are run with each assay. The controls are prepared in the same manner as described for the compounds of the invention. Ertapenem is included as a control for the serum protein binding assay.

### Plate Inoculation

All wells of microtiter plates are inoculated with (saline-diluted) culture using the MIC 2000 System, an automated plate inoculating device which delivers an inoculum of 1.5 TL per well. Plates are incubated at 35° C. in ambient air. An uninoculated plate is also incubated as a sterility check. Results are recorded after 22-24-hours' incubation. Plates were read to no growth. The MIC is defined as the lowest antimicrobial level which resulted in no growth after 22-24-hours' incubation.

The Compounds of formula I demonstrate antibacterial activity against various strains of S. aureus, E. faecalis, E. faecium, B. subtilis and S. pneumoniae. Compounds of formula I also demonstrate antibacterial activity against various species that are resistant to many known antibiotics such as methicillin-resistant S. aureus (MRSA), vancomycin-resistant Enterococcus sp. (VRE), multidrug-resistant E. faecium, macrolide-resistant S. aureus and S. epidermidis, and linezolid-resistant S. aureus and E. faecium. The minimum inhibitory concentration (MIC) values for these test strains range from 0.001 to 200 μg/mL. MICs are obtained in accordance to the NCCLS guidelines. Select compounds of this invention have been found to have minimum inhibitory concentration (MIC) values that are at least a 10 fold improvement over the compounds disclosed in P. Hmciar, et. al., J. Org. Chem. 2002, 67, 8789-8793 against tested strains. See Table 2 where compounds A and B (Examples 5 and 12 of claimed invention) were compared with compound C (example 7 of J. Org. Chem. 2002, 67, 8789-8793).

TABLE 2

| | | |
|---|---|---|
| Organism | Strain | MIC ug/mL |

Compound A

| | | |
|---|---|---|
| Enterococcus Faecalia | CLB 21560 | 0.015 |
| Staphylococcus Aureus | CL 5814 | 0.0038 |
| Staphylococcus Aureus | CL 8260 | 0.015 |
| Staphylococcus Aureus | MB 2865 | 0.0075 |

Compound B

| | | |
|---|---|---|
| Enterococcus Faecalia | CLB 21560 | 0.06 |
| Staphylococcus Aureus | CL 5814 | 0.0075 |
| Staphylococcus Aureus | CL 8260 | 0.03 |
| Staphylococcus Aureus | MB 2865 | 0.03 |

Compound C

| | | |
|---|---|---|
| Enterococcus Faecalia | CLB 21560 | 0.25375 |
| Staphylococcus Aureus | CL 5814 | 0.030475 |
| Staphylococcus Aureus | CL 8260 | 0.125475 |
| Staphylococcus Aureus | MB 2865 | 0.06 |

What is claimed is:

1. A compound of structural formula I: 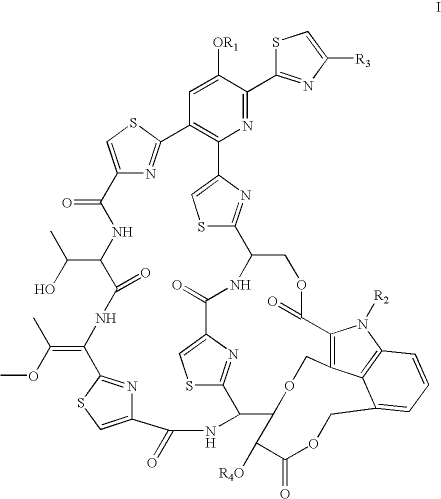
or a pharmaceutically acceptable salt, ester, enantiomer, diastereomer or mixture thereof,
wherein:
R independently represents hydrogen or C₁₋₁₂ alkyl;
R₁ represents hydrogen, C₁₋₆ alkyl, C₃₋₆ cycloalkyl, —(CH₂)ₙC₅₋₁₀ heterocyclyl, or —C(O)NR(CH₂)ₙC₅₋₁₀ heterocyclyl;
R₂ represents R₁ or OR₁;
R₃ represents —CH₂NR₅R₆ or C(O)H;
R₄ represents 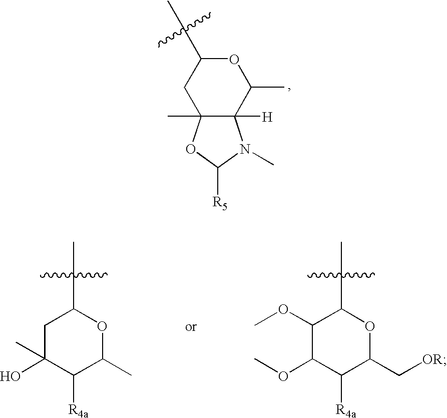
R₄ₐ represents N(R)₂;
R₅ and R₆ independently represent hydrogen, C₁₋₁₂ alkyl, —C(═NH)N(R₁)₂, —(CH₂)ₙC₅₋₁₀ heterocyclyl, —(CH₂)ₙNR₇R₈, —(CH₂)ₙNR(CH₂)ₙNR₇R₈, —(CH₂)ₙ(C₅₋₁₀ heterocyclyl, —(CH₂)ₙC(R)₂C₅₋₁₀ heterocyclyl, —(CH₂)ₙC₅₋₁₀ aryl, —(CH₂)ₙ(O(CH₂)₂)₁₋₆R_{9,} —(CHR)ₙNHC(O)(CH₂)ₙNR₇R₈, —(CH₂)ₙS(O)ₚ(CH₂)ₙC₅₋₁₀ heterocyclyl, —(CH₂)ₙCHR₇CF₃, —C(O)C₁₋₆ alkyl, —C(O)CF₃, —C(O)(C(R)₂)ₙNR₁R₇, —C(O)NR(CH₂)ₙC₅₋₁₀ heterocyclyl, —C(O)NR(CH₂)ₙNR₇R₈, —C(O)C(R)₂NHC(O)(CH₂)ₙNR₇R₈, —C(O)CHR₇(CH₂)ₙC(O)NR₁ R₁, —C(O)C(O)NR₁R₁, —C(O)(CH₂)ₙC₅₋₁₀ heterocyclyl, —C(R)₂(CH₂)ₙNHC(O)N(CH₂)ₙC₅₋₁₀ heterocyclyl, or —C(R)₂(CH₂)ₙOR, wherein said aryl and heterocyclyl are optionally substituted with one or more groups of R^{a}; said alkyl is optionally substituted with 1 to 6 hydroxy and/or optionally substituted by one or more groups of R^{a}; or
R₅ and R₆ together with the nitrogen atom they are attached form a 5 to 10 heterocyclic ring optionally containing 1 to 2 additional heteroatoms selected from the group consisting of N, S and O and optionally substituted with one or more groups of R^{a};
R₇ and R₈ independently represent hydrogen, hydroxyl, C₁₋₆ alkoxy, C₁₋₁₂ alkyl, —N(R)₂ —(CH₂)ₙNR₅R₆, —(CH₂)ₙC₅₋₁₀ heterocyclyl, —(CH₂)ₙC₆₋₁₀ aryl, —(CH₂)ₙOR, —C(O)R, —C(O)C₅₋₁₀ heterocyclyl, —C(O)NH(CH₂)ₙC₅₋₁₀ heterocyclyl, or —C(O)(CH₂)ₙN(R)_{2,} wherein said aryl and heterocyclyl are optionally substituted with one or more groups of R^{a}; said alkyl is optionally substituted with 1 to 6 hydroxyl and/or optionally substituted by one to more groups of R^{a}or
R₇ and R₈ together with the nitrogen atom they are attached form a 5 to 10 membered heterocyclic ring optionally containing 1 to 2 additional heteroatoms selected from the group consisting of N, S and O and optionally substituted with one or more groups of R^{a}; or
R₇ and R₈ together with the carbon atom they are attached form a 3 to 10 membered carbocyclic ring optionally and optionally substituted with one or more groups of R^{a};
R₉ represents hydrogen, C₁₋₆ alkyl, (CH₂)ₙC₅₋₁₀ heterocyclyl, —C(O)OR, CN, or OR, wherein said alkyl and heterocyclyl are optionally substituted with one or more groups of R^{a};
R^{a} represents hydrogen, halogen, (CH₂)ₙOR, CF_{3,} NHC(O)R, (CH₂)ₙC(O)OR, (CH₂)ₙC(O)NR₇R_{8,} (CH₂)ₙC₅₋₁₀ heterocyclyl, SO₂NR₅R_{6,} (CH₂)C₆₋₁₀ aryl, N(R)_{2,} NO_{2,} CN, —OP(O)(OR)_{2,} (C₁₋₆ alkyl)O—, (aryl)O—, (C₁₋₆ alkyl)S(O)₀₋₂—, or C₁₋₁₂ alkyl, wherein said alkyl, heterocyclyl, and aryl are optionally substituted with 1 to 4 groups selected from the group consisting of C₁₋₆ alkyl, (CH₂)ₙOR, (CH₂)ₙN(R)_{2,} and —O—; and n represent 0-6, and p represents 0, 1 or 2.

2. The compound according to claim 1 wherein R₁ represents hydrogen or —C₁₋₆ alkyl, and R₂ represents OH or OC₁₋₆ alkyl.

3. The compound according to claim 1 wherein R₃ represents —CH₂NR₅R_{6,} and R₅ and R₆ independently represent hydrogen, C₁₋₁₂ alkyl, —(CH₂)ₙC₅₋₁₀ heterocyclyl, —(CH₂)ₙNR₇R₈, —(CH₂)ₙNHC(O)(CH₂)ₙNR₇R₈, —C(O)C₁₋₆ alkyl, —C(O)(C(R)₂)ₙNR₁ R_{7,} —C(O)NR(CH₂)ₙC₅₋₁₀ heterocyclyl, or —C(O)(CH₂)ₙC₅₋₁₀ heterocyclyl, wherein said heterocyclyl is optionally substituted with one or more groups of R^{a}; said alkyl is optionally substituted with 1 to 6 hydroxy and/or optionally substituted by one or more groups of R^{a}.

4. The compound according to claim 1 wherein R₄ is: 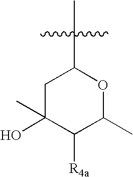
wherein R₄ₐ is N(R)₂ or N+(R)₂₀—.

5. The compound according to claim 4 wherein R₁ is H, R₂ is OH, R₄ₐ is —N(CH₃)₂, —NH₂, —NHCH_{3,} or —N+(CH₃)₂O—, and R₅ and R₆ independently represent hydrogen, C₁₋₁₂ alkyl, —(CH₂)ₙC₅₋₁₀ heterocyclyl, —(CH₂)ₙNR₇R₈, —(CH₂)ₙNHC(O)(CH₂)ₙNR₇R₈, —C(O)C₁₋₆ alkyl, —C(O)(C(R)₂)ₙNR₁ R₇, —C(O)NR(CH₂)ₙC₅₋₁₀ heterocyclyl, or —C(O)(CH₂)ₙC₅₋₁₀ heterocyclyl, wherein said heterocyclyl is optionally substituted with one or more groups of R^{a}; said alkyl is optionally substituted with 1 to 6 hydroxy and/or optionally substituted by one or more groups of R^{a}.

6. The compound according to claim 1 wherein R₄ represents 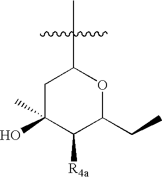
wherein R₄ₐ represents N(R)₂ or N+(R)₂)—.

7. The compound according to claim 1 represented by structural formula II: 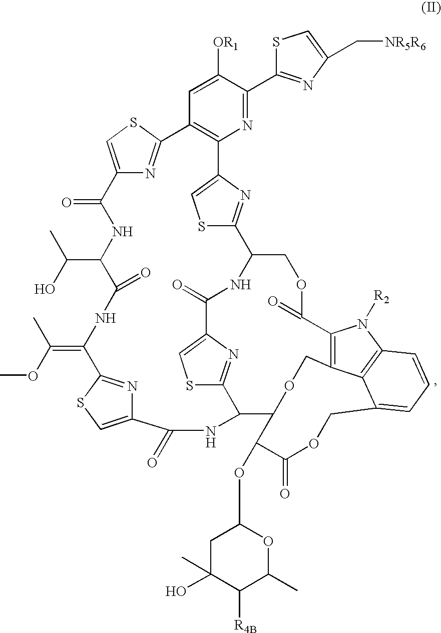
wherein R₁ is hydrogen, R₂ is OH, R₄ₐ is N(CH₃)₂, —NH_{2,} or —NHCH_{3,} and R₅ and R₆ independently represent hydrogen, C₁₋₁₂ alkyl, —(CH₂)ₙC₅₋₁₀ heterocyclyl, —(CH₂)ₙNR₇R₈, —(CH₂)ₙNHC(O)(CH₂)ₙNR₇R₈, —C(O)C₁₋₆ alkyl, —C(O)(C(R)₂)ₙNR₁R_{7,} —C(O)NR(CH₂)ₙC₅₋₁₀ heterocyclyl, or —C(O)(CH₂)ₙC₅₋₁₀ heterocyclyl, wherein said heterocyclyl is optionally substituted with one or more groups of R^{a}; said alkyl is optionally substituted with 1 to 6 hydroxy and/or optionally substituted by one or more groups of R^{a}.

8. The compound according to claim 1 represented by structural formula III: 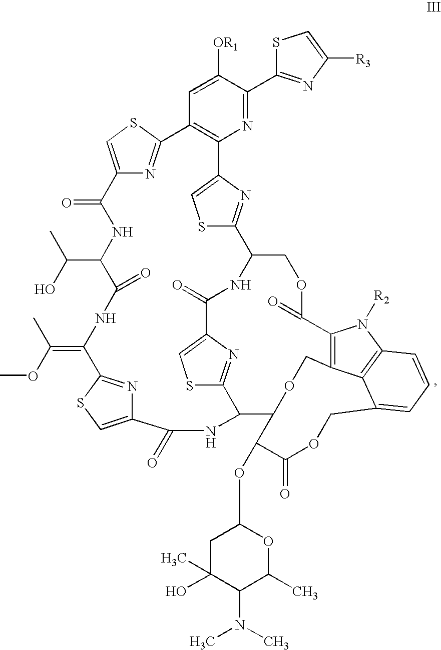
which is a compound selected from the group consisting of:

| Compound | R₁ | R₂ | R₃ |
|---|---|---|---|
| 1 | H | OH | 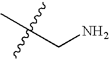 |
| 2 | H | OH | 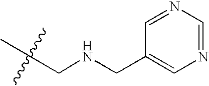 |
| 3 | H | OH | 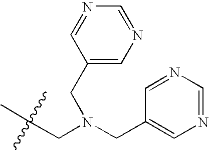 |
| 4 | H | OH | 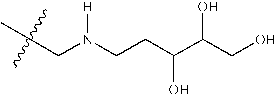 |
| 5 | H | OH | 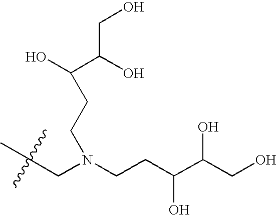 |
| 6 | H | OH | 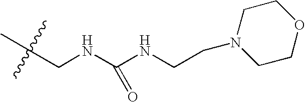 |
| 7 | H | 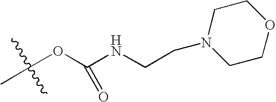 | 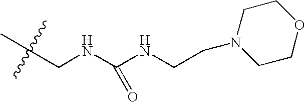 |
| 8 | H | OH | 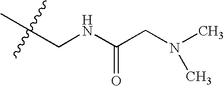 |
| 9 | H | OH | C(O)H |
| 10 | H | OH | 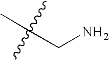 |
| 11 | H | H | 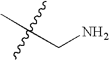 |
| 12 | 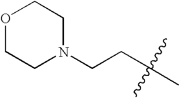 | —CH₂CH₃ | 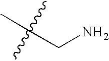 |
| 13 | CH₃ | OH | 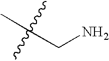 |
| 14 | H | OCH₃ | 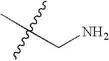 |
| 15 | H | OH | 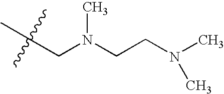 |
| 16 | H | OH | 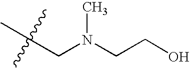 |
| 17 | H | OH | 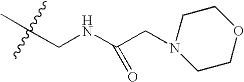 |
| 18 | H | OH | 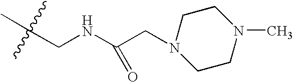 |
| 19 | H | OH | 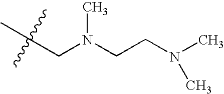 |
| 20 | H | OH | 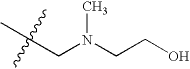 |
| 21 | H | OCH₃ | 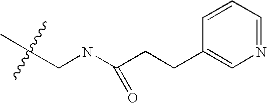 |
| 22 | H | OH | 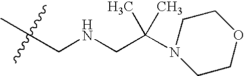 |
| 23 | H | H | 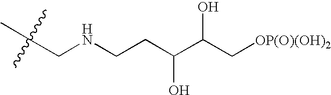 |
| 24 | H | OH | 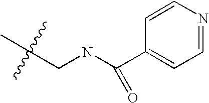 |
| 25 | H | OH | 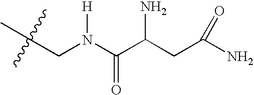 |
| 26 | H | OH | 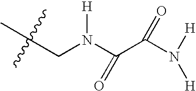 |
| 27 | 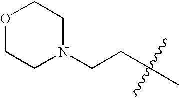 | H | 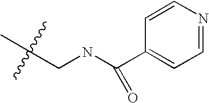 |
| 28 | 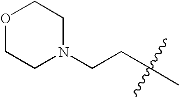 | H | 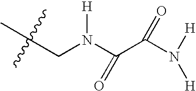 |
| 29 | H | OH | 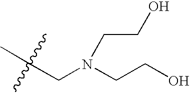 |
| 30 | H | OH | 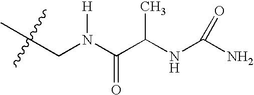 |
| 31 | H | OH | 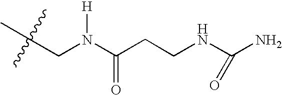 |
| 32 | H | OH | 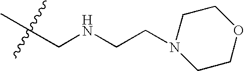 |
| 33 | H | H | 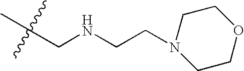 |
| 34 | H | OH | 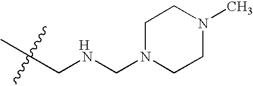 |
| 35 | H | OH | 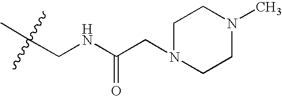 |
| 36 | H | H | 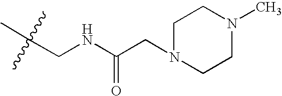 |
| 37 | H | OH | 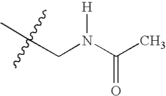 |
| 38 | H | OH | 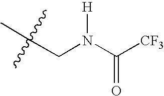 |
| 39 | H | OH | 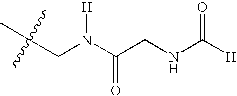 |
| 40 | H | OH | 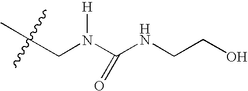 |
| 41 | H | OH | 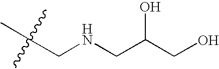 |
| 42 | H | OH | 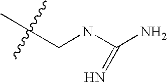 |
| 43 | H | OH | 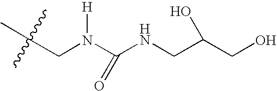 |
| 44 | 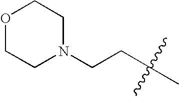 | OH | 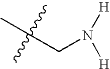 |
| 45 | 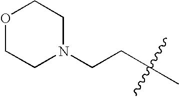 | OH | 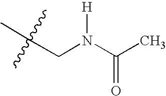 |
| 46 | H | OH | 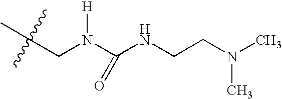 |
| 47 | H | OH | 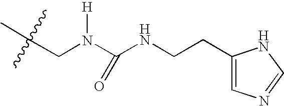 |
| 48 | H | OH | 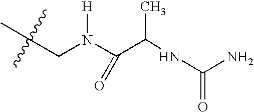 |
| 49 | H | OH | 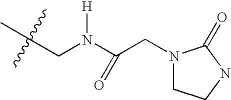 |
| 50 | H | OH | 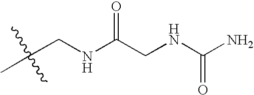 |

and pharmaceutically acceptable salts, esters, enantiomers, diastereomers, and mixtures thereof.

9. A pharmaceutical composition which is comprised of a compound in accordance with claim 1 and a pharmaceutically acceptable carrier.

10. A method of treating a bacterial infection in a mammal in need of such treatment which comprises administering to the mammal a compound of formula I of claim 1 in an amount effective to treat the infection.
